(19) United States
(12) Patent Application Publication
(10) Pub. No.: US 2012/0122076 A1
(43) Pub. Date: May 17, 2012

(54) PURIFICATION OF ANTIBODIES USING SIMULATED MOVING BED CHROMATOGRAPHY

(75) Inventors: Siu-Man Lau, Abbott Park, IL (US); Diane Dong, Abbott Park, IL (US); Stephen Lu, Abbott Park, IL (US)

(73) Assignee: Abbott Laboratories, Abbott Park, IL (US)

(21) Appl. No.: 13/234,938

(22) Filed: Sep. 16, 2011

Related U.S. Application Data
(60) Provisional application No. 61/384,620, filed on Sep. 20, 2010.

(51) Int. Cl. C12Q 1/00; C07K 16/00; C07K 1/18; C07K 14/575; C07K 1/20; C07K 1/22

(52) U.S. Cl. 435/4; 530/417; 530/413; 530/416; 530/399; 530/389.1; 530/388.15; 530/387.3

(57) ABSTRACT

The present invention relates to compositions and methods for the chromatographic purification of antibodies, such as monoclonal antibodies, employing improved simulated moving bed separation strategies and, in certain embodiments, Raman spectroscopy.

This application claims the benefit of the filing date of U.S. Ser. No. 61/384,620, filed on Sep. 20, 2010, the contents of which are incorporated herein by reference in their entirety.

### 1. INTRODUCTION

The present invention relates to compositions and methods for the chromatographic purification of antibodies, such as monoclonal antibodies (“mAbs”), employing improved simulated moving bed (“SMB”) separation strategies and, in certain embodiments, Raman spectroscopy.

### 2. BACKGROUND OF THE INVENTION

Protein purification strategies commonly employ one or more chromatographic separation steps in order to exclude host cell proteins (“HCPs”) from final purified protein preparations. Such chromatographic separation steps are traditionally performed in “batch mode”, where a single column packed with a particular chromatographic support is sequentially equilibrated, loaded, washed, eluted, and then regenerated. Because batch mode chromatography relies on loading the column only to the column's dynamic capacity rather than loading the column to its saturation capacity, each cycle of loading and separation makes use of only 30% to 50% of the column's actual binding capacity. Thus, batch mode separation requires the use of columns having two to three times more volume than would be needed if the columns were operated at their saturation capacity. By utilizing only 30%-50% of the column's actual binding capacity, batch mode chromatography therefore involves the use of significantly higher quantities of chromatographic separation supports and extends the time necessary to complete each cycle of loading and separation, which substantially raises the costs associated with protein purification. Furthermore, the use of columns having two to three times the volume that would be necessary if the separation was performed at saturation, leads to significant increases in the amount of equilibration, wash, and elution buffers employed in a single separation cycle, resulting in additional cost and time inefficiencies.

In light of the foregoing, there exists a need in the art for improved methods to more efficiently purify proteins, including therapeutic antibodies. The present invention addresses this need by incorporating improved simulated moving bed separation strategies into the purification of proteins.

### 3. SUMMARY OF THE INVENTION

In certain embodiments the present invention is directed towards methods for producing a host cell-protein (HCP) reduced target protein preparation from a sample mixture comprising a target protein and at least one HCP. In certain embodiments, the methods of the instant invention comprise contacting a target protein-containing sample mixture to a chromatography resin such that the resin is loaded to about 50%400%, including greater than about 50%, greater than about 60%, greater than about 70%, greater than about 80%, and greater than about 90%, of its saturated binding capacity, and collecting a chromatographic sample, wherein said chromatographic sample comprises said HCP-reduced target protein preparation. In certain of such embodiments, Raman spectroscopy is employed in order to monitor and/or determine the composition of one or more of the multi-component mixtures involved in the production of such HCP-reduced target protein preparations.

Certain embodiments of the present invention are directed to the production of HCP-reduced target protein preparations that comprise contacting a target protein-containing sample mixture to a chromatography resin such that the resin is loaded to between about 50%-100%, including greater than about 50%, greater than about 60%, greater than about 70%, greater than about 80%, and greater than about 90%, of its saturated binding capacity, and collecting a chromatographic sample, wherein said chromatographic sample comprises said HCP-reduced target protein preparation and the chromatographic resin selected from the group consisting of affinity chromatographic resin, ion exchange chromatographic resin, and hydrophobic interaction chromatographic resin. In certain of such embodiments, Raman spectroscopy is employed in order to monitor and/or determine the composition of one or more of the multi-component mixtures involved in the production of such HCP-reduced target protein preparations.

Certain embodiments of the present invention are directed to the production of HCP-reduced target protein preparations that comprise contacting a target protein-containing sample mixture to a chromatography resin such that the resin is loaded to about 50%400%, including greater than about 50%, greater than about 60%, greater than about 70%, greater than about 80%, and greater than about 90%, of its saturated binding capacity, and collecting a chromatographic sample, wherein said chromatographic sample comprises said HCP-reduced target protein preparation and the target protein is selected from the group consisting of: enzymes; peptide hormones; polyclonal antibodies; human monoclonal antibodies; humanized monoclonal antibodies; chimeric monoclonal antibodies; single chain antibodies; Fab antibody fragments; F(ab′)₂ antibody fragments; Fd antibody fragments; Fv antibody fragments; isolated CDRs; diabodies; DVDs, and immunoadhesions. In certain of such embodiments, Raman spectroscopy is employed in order to monitor and/or determine the composition of one or more of the multi-component mixtures involved in the production of such HCP-reduced target protein preparations.

Certain embodiments of the present invention are directed to the production of HCP-reduced target protein preparations that comprise contacting a target protein-containing sample mixture to a chromatography resin such that the resin is loaded to about 50%-100%, including greater than about 50%, greater than about 60%, greater than about 70%, greater than about 80%, and greater than about 90%, of its saturated binding capacity, and collecting a chromatographic sample, wherein said chromatographic sample comprises said HCP-reduced target protein preparation and the chromatography resin is packed into a series of fluidly-connected columns separated by fluid conduits, wherein the number of fluidly connected columns is selected from the group consisting of: 2, 3, 4, 5, 6, 7, 8, 9, 10, 11, and 12 individual columns. In certain of such embodiments, Raman spectroscopy is employed in order to monitor and/or determine the composition of one or more of the multi-component mixtures involved in the production of such HCP-reduced target protein preparations.

Certain embodiments of the present invention are directed to the production of HCP-reduced target protein preparations that comprise contacting a target protein-containing sample mixture to a chromatography resin such that the resin is loaded to about 50%400%, including greater than about 50%, greater than about 60%, greater than about 70%, greater than about 80%, and greater than about 90%, of its saturated binding capacity, and collecting a chromatographic sample, wherein said chromatographic sample comprises said HCP-reduced target protein preparation and the chromatography resin is packed into a series of at least 2 fluidly-connected columns separated by fluid conduits, wherein the columns are separated by fluid conduits that permit the introduction buffers, such as equilibration, wash, and elution buffers, as well as the withdrawal of eluates. In certain of such embodiments, Raman spectroscopy is employed in order to monitor and/or determine the composition of one or more of the multi-component mixtures involved in the production of such HCP-reduced target protein preparations.

Certain embodiments of the present invention are directed to the production of HCP-reduced target protein preparations that comprise contacting a target protein-containing sample mixture to a chromatography resin such that the resin is loaded to about 50%400%, including greater than about 50%, greater than about 60%, greater than about 70%, greater than about 80%, and greater than about 90%, of its saturated binding capacity, and collecting a chromatographic sample, wherein said chromatographic sample comprises said HCP-reduced target protein preparation and the sample mixture is contacted to the chromatography resin in order to obtain a residence time selected from a range of about 0.5 to about 12 minutes, in one embodiment it can be selected from the group consisting of up to about 0.5, up to about 1, up to about 2, up to about 3, up to about 4, up to about 5, up to about 6, up to about 7, up to about 8, up to about 9, up to about 10, up to about 11, and up to about 12 minutes. In certain of such embodiments, Raman spectroscopy is employed in order to monitor and/or determine the composition of one or more of the multi-component mixtures involved in the production of such HCP-reduced target protein preparations.

Certain embodiments of the present invention are directed to the production of HCP-reduced target protein preparations that comprise contacting a target protein-containing sample mixture to a chromatography resin such that the resin is loaded to about 50%400%, including greater than about 50%, greater than about 60%, greater than about 70%, greater than about 80%, and greater than about 90%, of its saturated binding capacity, and collecting a chromatographic sample, wherein said chromatographic sample comprises said HCP-reduced target protein preparation and the method further comprises the steps of equilibrating the chromatographic resin prior to contact with the sample mixture and washing the chromatographic resin after contact with the sample mixture, where the equilibration and wash buffers are identical buffers. In certain of such embodiments, Raman spectroscopy is employed in order to monitor and/or determine the composition of one or more of the multi-component mixtures involved in the production of such HCP-reduced target protein preparations.

Certain embodiments of the present invention are directed to the production of HCP-reduced target protein preparations that comprise contacting a target protein-containing sample mixture to a chromatography resin such that the resin is loaded to about 50%400%, including greater than about 50%, greater than about 60%, greater than about 70%, greater than about 80%, and greater than about 90%, of its saturated binding capacity, and collecting a chromatographic sample, wherein said chromatographic sample comprises said HCP-reduced target protein preparation and the method further comprises chromatography resin wash, and regeneration steps, where such steps can be calculated and programmed in order to maintain the step of contacting the sample to the chromatography resin to be from about 20% to about 80% of the time of the process, in one particular embodiment it is about 50%. In certain of such embodiments, Raman spectroscopy is employed in order to monitor and/or determine the composition of one or more of the multi-component mixtures involved in the production of such HCP-reduced target protein preparations.

### 4. BRIEF DESCRIPTION OF THE FIGURES

FIG. 1. depicts a conventional chromatography flow diagram vs. a simulated moving bed chromatography flow diagram.

FIG. 2. depicts a conventional chromatography flow diagram.

FIG. 3. depicts a simulated moving bed chromatography flow diagram.

FIG. 4. depicts a chromatogram reflecting the results of the mAb X simulated moving bed chromatography case study.

FIG. 5. depicts the product recovery and product quality analysis for the mAb X simulated moving bed chromatography case study.

FIG. 6. depicts a chromatogram reflecting the results of the mAb Y simulated moving bed chromatography case study.

FIG. 7. depicts the product recovery and product quality analysis for the mAb Y simulated moving bed chromatography case study.

FIG. 8. depicts the mAb X % breakthrough analysis relating to the mAb X simulated moving bed chromatography case study.

FIG. 9. depicts the mAb Y % breakthrough analysis relating to the mAb Y simulated moving bed chromatography case study.

FIG. 10. depicts a pilot scale simulated moving bed chromatography flow diagram.

FIG. 11. depicts the Raman Spectra of 3 Component (arginine/citric acid/trehalose) buffer system that includes an amino acid, a pH buffer species, and a sugar. This plot was generated using Umetrics SIMCA P+ V 12.0.1.0. The X axis is the datapoint number. Each data point is a Raman Shift wavenumber. It could be replotted with Raman Shift wavenumber (cm⁻¹) on the X axis. The data starts with wavenumber 1800 (=Num 0) to 800 (=Num 1000). The Raman spectral raw data is in units of Intensity (related to the number of scattered photons). This Figure shows the mean centered spectral data of the three individual components (in water). The average value of the spectra is 0. The other values are relative to that, probably in standard deviations from the mean.

FIG. 12. depicts a comparison of actual vs. predicted concentration for a 3 component buffer system (arginine/citric acid/trehalose) with random values. This Figure was created using the existing model to predict the concentrations of new solutions. The x and y-axis are concentrations (mM).

FIG. 13. depicts a comparison of actual vs. predicted concentration for 3 component buffer system (arginine/citric acid/trehalose) by individual component.

FIG. 14. depicts a pure component raw spectra of 4 component buffer system (mannitol/methionine/histidine/Tween™). The y-axis is spectral intensity, the x-axis is wave number cm-1.

FIG. 15. depicts a pure component raw spectra of 4 component buffer system (mannitol/methionine/histidine/Tween™) The y-axis is spectral intensity, the x-axis is wave number cm-1. FIG. 5 is an more detailed view of the spectra shown in FIG. 4, in which the “fingerprint” region has been expanded.

FIG. 16. depicts a pure component SNV/DYDX/Mean Center spectra of 4 component buffer system (mannitol/methionine/histidine/Tween™). The data shown in FIG. 6 is based on the same data shown in FIGS. 4-5, after all preprocessing: standard normal variate (SNV) for intensity normalization, 1st derivative for base line normalization, and mean centering for scaling.

FIG. 17. depicts a comparison of actual vs. predicted concentration for 4 component buffer system (mannitol/methionine/histidine/Tween™) with random values. This was created using the existing model to predict the concentrations of new solutions.

FIG. 18. depicts a comparison of actual vs. predicted concentration for 3 component buffer system (mannitol/methionine/histidine/Tween™) by individual component.

FIG. 19. depicts a pure component raw spectra for 3 component buffer system with protein (mannitol/methionine/histidine/adalimumab) Raw spectra showing Raman intensity.

FIG. 20. depicts a pure component raw spectra for 3 component buffer system with protein (mannitol/methionine/histidine/adalimumab), with the fingerprint region (800-1700 cm-1) shown in detail.

FIG. 21. depicts a pure component SNV/DYDX/Mean Center—3 component buffer system with protein. The data shown in FIG. 11 is based on the same data shown in FIGS. 9-10, after all preprocessing: standard normal variate (SNV) for intensity normalization, 1st derivative for base line normalization, and mean centering for scaling.

FIG. 22. depicts a comparison of actual vs. predicted concentration for 3 component buffer system with protein by individual component.

FIG. 23. depicts an adalimumab purification process that employs Raman Spectroscopy as part of process and/or quality control.

FIG. 24. depicts on-line Raman concentration predictions of a diafiltration process involving a three component mixture of buffer, sugar, and amino acid (methionine/mannitol/histidine).

FIG. 25. depicts repeated diafiltration process involving a three component mixture of buffer, sugar, and amino acid (methionine/mannitol/histidine). Additional data points included for increased resolution.

FIG. 26. depicts a Raman calibration of sugar (mannitol)/protein (adalimumab) solution.

FIG. 27. depicts on-line Raman concentration predictions of a diafiltration buffer exchange process where antibody in water is replaced with a mannitol solution to provide a sugar/protein (mannitol/adalimumab) solution. The buffer exchanged is followed by protein concentration.

FIG. 28. depicts a repeat of the experiment depicted in FIG. 27, where the protein concentration phase is extended to 180 g/L.

FIG. 29. depicts a Raman calibration of histidine and adalimumab solutions.

FIG. 30. depicts on-line Raman concentration predictions of a diafiltration buffer exchange process where protein in water is replaced with a histidine solution. The histidine exchanged is followed by adalimumab concentration.

FIG. 31A-C. depicts a comparison of actual vs. predicted concentration for 2 component buffer system with protein by individual component: A. Tris concentration; B. Acetate concentration; and C. Adalimumab concentration.

FIG. 32A-B. depicts a comparison of actual vs. predicted concentration for 1 component buffer system with protein by individual component: A. Tween™ concentration; and B. Adalimumab concentration.

FIG. 33. depicts the conditions of employed when two antibodies (D2E7 and ABT-874) were separately aggregated using photo induced cross-linking of unmodified proteins (PICUP). The antibodies were exposed to the aggregating light source from 0-4 hours.

FIG. 34. depicts the size exclusion chromatographic results of the cross-linking outlined in FIG. 33.

FIG. 35. depicts Raman spectroscopy and the spectra modeled using principal component analysis of D2E7 samples, indicating that aggregated samples have distinct principal component scores and can be discriminated from aggregates using Raman spectroscopy

FIG. 36. depicts Raman spectroscopy and the spectra modeled using principal component analysis of ABT-874 samples, indicating that aggregated samples have distinct principal component scores and can be discriminated from aggregates using Raman spectroscopy.

FIG. 37A-B. depicts Raman spectroscopy and the spectra modeled using partial least squares analysis of (A) D2E7 samples and (B) ABT-974 samples, indicating some correlation between Raman spectroscopy results and the SEC measurements.

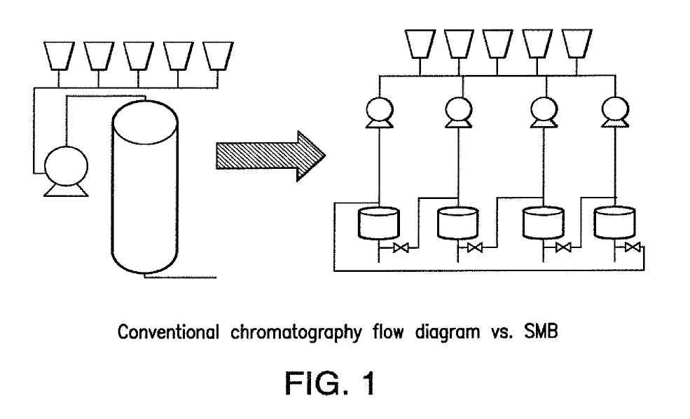

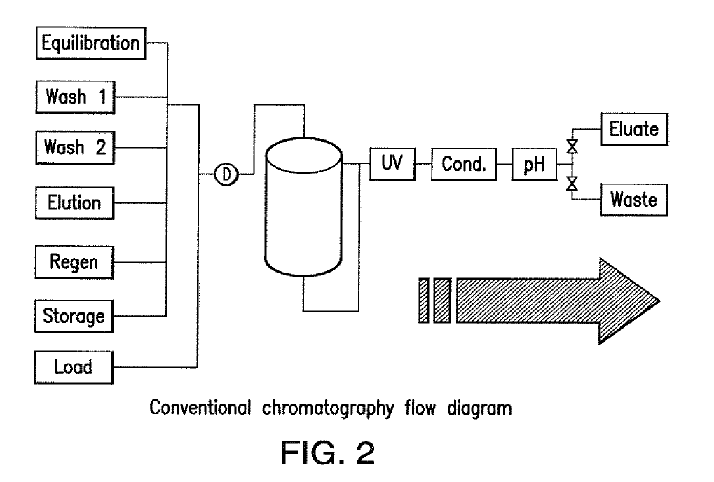

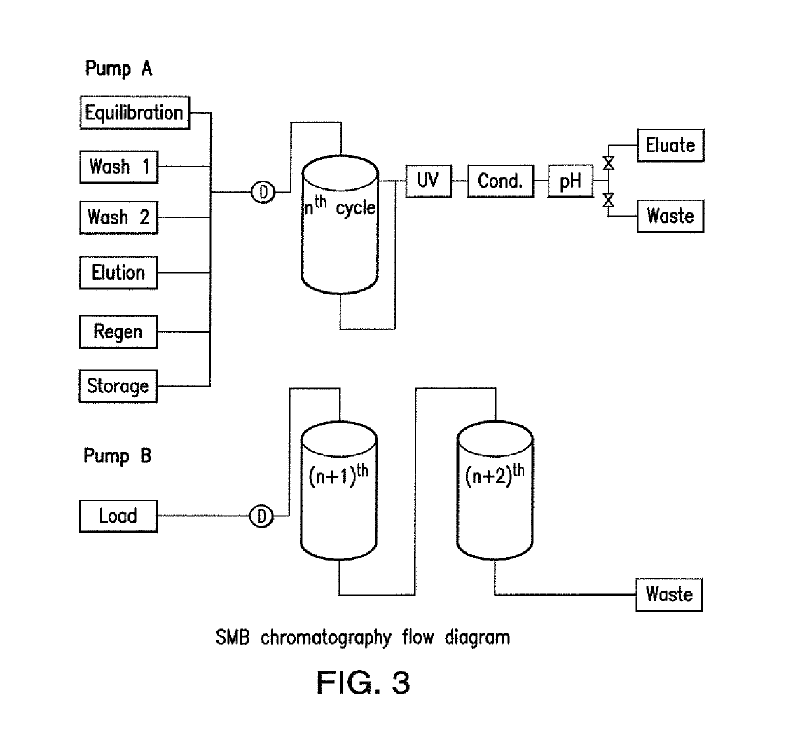

### 5. DETAILED DESCRIPTION OF THE INVENTION

The present invention relates to compositions and methods for chromatographic purification of antibodies, such as monoclonal antibodies, employing improved simulated moving bed separation strategies. For clarity and not by way of limitation, this detailed description is divided into the following sub-portions:

5.1. Definitions

5.2. Antibody Generation;

5.3. Antibody Production;

5.4. Antibody Purification;

5.5 Exemplary Purification Strategies; and

5.6 Raman Spectroscopy.

### 5.1. DEFINITIONS

The term “antibody” includes an immunoglobulin molecule comprised of four polypeptide chains, two heavy (H) chains and two light (L) chains inter-connected by disulfide bonds. Each heavy chain is comprised of a heavy chain variable region (abbreviated herein as HCVR or VH) and a heavy chain constant region (CH). The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

The term “antigen-binding portion” of an antibody (or “antibody portion”) includes fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include (i) a Fab fragment, a monovalent fragment comprising the VL, VH, CL and CH1 domains; (ii) a F(ab′)2 fragment, a bivalent fragment comprising two Fab fragments linked by a disulfide bridge at the hinge region; (iii) a Fd fragment comprising the VH and CH1 domains; (iv) a Fv fragment comprising the VL and VH domains of a single arm of an antibody, (v) a dAb fragment (Ward et al., (1989) Nature 341:544-546, the entire teaching of which is incorporated herein by reference), which comprises a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al. (1988) Science 242:423-426; and Huston et al. (1988) Proc. Natl. Acad. Sci. USA 85:5879-5883, the entire teachings of which are incorporated herein by reference). Such single chain antibodies are also intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see, e.g., Holliger, P., et al. (1993) Proc. Natl. Acad. Sci. USA 90:6444-6448; Poljak, R. J., et al. (1994) Structure 2:1121-1123, the entire teachings of which are incorporated herein by reference). Still further, an antibody or antigen-binding portion thereof may be part of a larger immunoadhesion molecule, formed by covalent or non-covalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101, the entire teaching of which is incorporated herein by reference) and use of a cysteine residue, a marker peptide and a C-terminal polyhistidine tag to make bivalent and biotinylated scFv molecules (Kipriyanov, S. M., et al. (1994) Mol. Immunol. 31:1047-1058, the entire teaching of which is incorporated herein by reference). Antibody portions, such as Fab and F(ab′)2 fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein. In one aspect, the antigen binding portions are complete domains or pairs of complete domains.

The terms “Kabat numbering”, “Kabat definitions” and “Kabat labeling” are used interchangeably herein. These terms, which are recognized in the art, refer to a system of numbering amino acid residues which are more variable (i.e., hypervariable) than other amino acid residues in the heavy and light chain variable regions of an antibody, or an antigen binding portion thereof (Kabat et al. (1971) Ann. NY Acad, Sci. 190:382-391 and, Kabat, E. A., et al. (1991) Sequences of Proteins of Immunological Interest, Fifth Edition, U.S. Department of Health and Human Services, NIH Publication No. 91-3242, the entire teachings of which are incorporated herein by reference). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3

The term “human antibody” includes antibodies having variable and constant regions corresponding to human germline immunoglobulin sequences as described by Kabat et al. (See Kabat, et al. (1991) Sequences of proteins of Immunological Interest, Fifth Edition, U.S. Department of Health and Human Services, NIH Publication No. 91-3242). The human antibodies of the invention may include amino acid residues not encoded by human germline immunoglobulin sequences (e.g., mutations introduced by random or site-specific mutagenesis in vitro or by somatic mutation in vivo), e.g., in the CDRs and in particular CDR3. The mutations can be introduced using a selective mutagenesis approach. The human antibody can have at least one position replaced with an amino acid residue, e.g., an activity enhancing amino acid residue which is not encoded by the human germline immunoglobulin sequence. The human antibody can have up to twenty positions replaced with amino acid residues which are not part of the human germline immunoglobulin sequence. In other embodiments, up to ten, up to five, up to three or up to two positions are replaced. In one embodiment, these replacements are within the CDR regions. However, the term “human antibody”, as used herein, is not intended to include antibodies in which CDR sequences derived from the germline of another mammalian species, such as a mouse, have been grafted onto human framework sequences.

The phrase “recombinant human antibody” includes human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse) that is transgenic for human immunoglobulin genes (see, e.g., Taylor, L. D., et al. (1992) Nucl. Acids Res. 20:6287-6295, the entire teaching of which is incorporated herein by reference) or antibodies prepared, expressed, created or isolated by any other means that involves splicing of human immunoglobulin gene sequences to other DNA sequences. Such recombinant human antibodies have variable and constant regions derived from human germline immunoglobulin sequences (see, Kabat, E. A., et al. (1991) Sequences of Proteins of Immunological Interest, Fifth Edition, U.S. Department of Health and Human Services, NIH Publication No. 91-3242). In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. In certain embodiments, however, such recombinant antibodies are the result of selective mutagenesis approach or back-mutation or both.

An “isolated antibody” includes an antibody that is substantially free of other antibodies having different antigenic specificities (e.g., an isolated antibody that specifically binds a particular target is substantially free of antibodies that specifically bind antigens other than the specified target). An isolated antibody that specifically binds a particular human target may bind the same target from other species. Moreover, an isolated antibody may be substantially free of other cellular material and/or chemicals.

The term “Koff”, as used herein, is intended to refer to the off rate constant for dissociation of an antibody from the antibody/antigen complex.

The term “Kd”, as used herein, is intended to refer to the dissociation constant of a particular antibody-antigen interaction.

The phrase “nucleic acid molecule” includes DNA molecules and RNA molecules. A nucleic acid molecule may be single-stranded or double-stranded, but in one aspect is double-stranded DNA.

The phrase “isolated nucleic acid molecule,” as used herein in reference to nucleic acids encoding antibodies or antibody portions (e.g., VH, VL, CDR3), e.g. those that bind a particular target and includes a nucleic acid molecule in which the nucleotide sequences encoding the antibody or antibody portion are free of other nucleotide sequences encoding antibodies or antibody portions that bind antigens other than the particular target, which other sequences may naturally flank the nucleic acid in human genomic DNA. The phrase “isolated nucleic acid molecule” is also intended to include sequences encoding bivalent, bispecific antibodies, such as diabodies in which VH and VL regions contain no other sequences other than the sequences of the diabody.

The phrase “recombinant host cell” (or simply “host cell”) includes a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

The term “about”, as used herein, is intended to refer to ranges of approximately 10-20% greater than or less than the referenced value. In certain circumstances, one of skill in the art will recognize that, due to the nature of the referenced value, the term “about” can mean more or less than a 10-20% deviation from that value.

“Chromatography”, as used herein, refers to analytical techniques used for the separation of target molecules of interest from a mixture of molecules, and relies upon selective attraction among components of the mixture to a solid phase. Examples include affinity chromatography, ion exchange chromatography, size exclusion chromatography, and hydrophobic interaction chromatography.

“Purified” when referring to a target molecule of interest in a mixture indicates that its relative concentration (weight of target divided by the weight of all components or fractions in the mixture) is increased by at least 20%. In one series of embodiments, the relative concentration is increased by at least about 40%, about 50%, about 60%, about 75%, about 100%, about 150%, or about 200%. A target molecule of interest can also be said to be purified when the relative concentration of components from which it is purified (weight of component or fraction from which it is purified divided by the weight of all components or fractions in the mixture) is decreased by at least about 20%, about 40%, about 50%, about 60%, about 75%, about 85%, about 95%, about 98% or about 100%. In still another series of embodiments, the target molecule of interest is purified to a relative concentration of at least about 50%, about 65%, about 75%, about 85%, about 90%, about 97%, about 98%, or about 99%. When a target molecule of interest in one embodiment is “separated” from other components or fractions, it will be understood that in other embodiments the component or fraction is “purified” at levels provided herein.

### 5.2. ANTIBODY GENERATION

The term “antibody” as used in this section refers to an intact antibody or an antigen binding fragment thereof.

The antibodies of the present disclosure can be generated by a variety of techniques, including immunization of an animal with the antigen of interest followed by conventional monoclonal antibody methodologies e.g., the standard somatic cell hybridization technique of Kohler and Milstein (1975) Nature 256: 495. Although somatic cell hybridization procedures are typical, in principle, other techniques for producing monoclonal antibody can be employed e.g., viral or oncogenic transformation of B lymphocytes.

One typical animal system for preparing hybridomas is the murine system. Hybridoma production is a very well-established procedure. Immunization protocols and techniques for isolation of immunized splenocytes for fusion are known in the art. Fusion partners (e.g., murine myeloma cells) and fusion procedures are also known.

An antibody typically can be a human, a chimeric, or a humanized antibody. Chimeric or humanized antibodies of the present disclosure can be prepared based on the sequence of a non-human monoclonal antibody prepared as described above. DNA encoding the heavy and light chain immunoglobulins can be obtained from the non-human hybridoma of interest and engineered to contain non-murine (e.g., human) immunoglobulin sequences using standard molecular biology techniques. For example, to create a chimeric antibody, murine variable regions can be linked to human constant regions using methods known in the art (see e.g., U.S. Pat. No. 4,816,567 to Cabilly et al.). To create a humanized antibody, murine CDR regions can be inserted into a human framework using methods known in the art (see e.g., U.S. Pat. No. 5,225,539 to Winter, and U.S. Pat. Nos. 5,530,101; 5,585,089; 5,693,762 and 6,180,370 to Queen et al.).

In one non-limiting embodiment, the antibodies of this disclosure are human monoclonal antibodies. Such human monoclonal antibodies can be generated using transgenic or transchromosomic mice carrying parts of the human immune system rather than the mouse system. These transgenic and transchromosomic mice include mice referred to herein as the HuMAb Mouse® (Medarex, Inc.), KM Mouse® (Medarex, Inc.), and XenoMouse® (Amgen).

Moreover, alternative transchromosomic animal systems expressing human immunoglobulin genes are available in the art and can be used to raise antibodies of the disclosure. For example, mice carrying both a human heavy chain transchromosome and a human light chain tranchromosome, referred to as “TC mice” can be used; such mice are described in Tomizuka et al. (2000) Proc. Natl. Acad. Sci. USA 97:722-727. Furthermore, cows carrying human heavy and light chain transchromosomes have been described in the art (e.g., Kuroiwa et al. (2002) Nature Biotechnology 20:889-894 and PCT application No. WO 2002/092812) and can be used to raise the antibodies of this disclosure.

Recombinant human antibodies of the invention can be isolated by screening of a recombinant combinatorial antibody library, e.g., a scFv phage display library, prepared using human VL and VH cDNAs prepared from mRNA derived from human lymphocytes. Methodologies for preparing and screening such libraries are known in the art. In addition to commercially available kits for generating phage display libraries (e.g., the Pharmacia Recombinant Phage Antibody System, catalog no. 27-9400-01; and the Stratagene SurfZAP™ phage display kit, catalog no. 240612, the entire teachings of which are incorporated herein), examples of methods and reagents particularly amenable for use in generating and screening antibody display libraries can be found in, e.g., Ladner et al. U.S. Pat. No. 5,223,409; Kang et al. PCT Publication No. WO 92/18619; Dower et al. PCT Publication No. WO 91/17271; Winter et al. PCT Publication No. WO 92/20791; Markland et al. PCT Publication No. WO 92/15679; Breitling et al. PCT Publication No. WO 93/01288; McCafferty et al. PCT Publication No. WO 92/01047; Garrard et al. PCT Publication No. WO 92/09690; Fuchs et al. (1991) Bio/Technology 9:1370-1372; Hay et al. (1992) Hum Antibod Hybridomas 3:81-85; Huse et al. (1989) Science 246:1275-1281; McCafferty et al., Nature (1990) 348:552-554; Griffiths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrard et al. (1991) Bio/Technology 9:1373-1377; Hoogenboom et al. (1991) Nuc Acid Res 19:4133-4137; and Barbas et al. (1991) PNAS 88:7978-7982; the entire teachings of which are incorporated herein.

Human monoclonal antibodies of this disclosure can also be prepared using SCID mice into which human immune cells have been reconstituted such that a human antibody response can be generated upon immunization. Such mice are described in, for example, U.S. Pat. Nos. 5,476,996 and 5,698,767 to Wilson et al.

In yet another embodiment of the invention, antibodies or fragments thereof, can be altered wherein the constant region of the antibody is modified to reduce at least one constant region-mediated biological effector function relative to an unmodified antibody. To modify an antibody of the invention such that it exhibits reduced binding to the Fc receptor, the immunoglobulin constant region segment of the antibody can be mutated at particular regions necessary for Fc receptor (FcR) interactions (see, e.g., Canfield and Morrison (1991) J. Exp. Med. 173:1483-1491; and Lund et al. (1991) J. of Immunol. 147:2657-2662, the entire teachings of which are incorporated herein). Reduction in FcR binding ability of the antibody may also reduce other effector functions which rely on FcR interactions, such as opsonization and phagocytosis and antigen-dependent cellular cytotoxicity.

### 5.3. ANTIBODY PRODUCTION

To express an antibody of the invention, DNAs encoding partial or full-length light and heavy chains are inserted into one or more expression vector such that the genes are operatively linked to transcriptional and translational control sequences. (See, e.g., U.S. Pat. No. 6,914,128, the entire teaching of which is incorporated herein by reference.) In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into a separate vector or, more typically, both genes are inserted into the same expression vector. The antibody genes are inserted into an expression vector by standard methods (e.g., ligation of complementary restriction sites on the antibody gene fragment and vector, or blunt end ligation if no restriction sites are present). Prior to insertion of the antibody or antibody-related light or heavy chain sequences, the expression vector may already carry antibody constant region sequences. For example, one approach to converting particular VH and VL sequences to full-length antibody genes is to insert them into expression vectors already encoding heavy chain constant and light chain constant regions, respectively, such that the VH segment is operatively linked to the CH segment(s) within the vector and the VL segment is operatively linked to the CL segment within the vector. Additionally or alternatively, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, a recombinant expression vector of the invention can carry one or more regulatory sequence that controls the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, e.g., in Goeddel; Gene Expression Technology: Methods in Enzymology 185, Academic Press, San Diego, Calif. (1990), the entire teaching of which is incorporated herein by reference. It will be appreciated by those skilled in the art that the design of the expression vector, including the selection of regulatory sequences may depend on such factors as the choice of the host cell to be transformed, the level of expression of protein desired, etc. Suitable regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. For further description of viral regulatory elements, and sequences thereof, see, e.g., U.S. Pat. No. 5,168,062 by Stinski, U.S. Pat. No. 4,510,245 by Bell et al. and U.S. Pat. No. 4,968,615 by Schaffner et al., the entire teachings of which are incorporated herein by reference.

In addition to the antibody chain genes and regulatory sequences, a recombinant expression vector of the invention may carry one or more additional sequences, such as a sequence that regulates replication of the vector in host cells (e.g., origins of replication) and/or a selectable marker gene. The selectable marker gene facilitates selection of host cells into which the vector has been introduced (see e.g., U.S. Pat. Nos. 4,399,216, 4,634,665 and 5,179,017, all by Axel et al., the entire teachings of which are incorporated herein by reference). For example, typically the selectable marker gene confers resistance to drugs, such as G418, hygromycin or methotrexate, on a host cell into which the vector has been introduced. Suitable selectable marker genes include the dihydrofolate reductase (DHFR) gene (for use in dhfr-host cells with methotrexate selection/amplification) and the neo gene (for G418 selection).

An antibody, or antibody portion, of the invention can be prepared by recombinant expression of immunoglobulin light and heavy chain genes in a host cell. To express an antibody recombinantly, a host cell is transfected with one or more recombinant expression vectors carrying DNA fragments encoding the immunoglobulin light and heavy chains of the antibody such that the light and heavy chains are expressed in the host cell and secreted into the medium in which the host cells are cultured, from which medium the antibodies can be recovered. Standard recombinant DNA methodologies are used to obtain antibody heavy and light chain genes, incorporate these genes into recombinant expression vectors and introduce the vectors into host cells, such as those described in Sambrook, Fritsch and Maniatis (eds), Molecular Cloning; A Laboratory Manual, Second Edition, Cold Spring Harbor, N.Y., (1989), Ausubel et al. (eds.) Current Protocols in Molecular Biology, Greene Publishing Associates, (1989) and in U.S. Pat. Nos. 4,816,397 & 6,914,128, the entire teachings of which are incorporated herein.

For expression of the light and heavy chains, the expression vector(s) encoding the heavy and light chains is (are) transfected into a host cell by standard techniques. The various forms of the teen “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the antibodies of the invention in either prokaryotic or eukaryotic host cells, expression of antibodies in eukaryotic cells, such as mammalian host cells, is suitable because such eukaryotic cells, and in particular mammalian cells, are more likely than prokaryotic cells to assemble and secrete a properly folded and immunologically active antibody. Prokaryotic expression of antibody genes has been reported to be ineffective for production of high yields of active antibody (Boss and Wood (1985) Immunology Today 6:12-13, the entire teaching of which is incorporated herein by reference).

Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, e.g., Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published Apr. 12, 1989), Pseudomonas such as P. aeruginosa, and Streptomyces. One suitable E. coli cloning host is E. coli 294 (ATCC 31,446), although other strains such as E. coli B, E. coli X1776 (ATCC 31,537), and E. coli W3110 (ATCC 27,325) are suitable. These examples are illustrative rather than limiting.

In addition to prokaryotes, eukaryotic microbes such as filamentous fungi or yeast are suitable cloning or expression hosts for polypeptide encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated antibodies are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

Suitable mammalian host cells for expressing the recombinant antibodies of the invention include Chinese Hamster Ovary (CHO cells) (including dhfr-CHO cells, described in Urlaub and Chasin, (1980) PNAS USA 77:4216-4220, used with a DHFR selectable marker, e.g., as described in Kaufman and Sharp (1982) Mol. Biol. 159:601-621, the entire teachings of which are incorporated herein by reference), NS0 myeloma cells, COS cells and SP2 cells. When recombinant expression vectors encoding antibody genes are introduced into mammalian host cells, the antibodies are produced by culturing the host cells for a period of time sufficient to allow for expression of the antibody in the host cells or secretion of the antibody into the culture medium in which the host cells are grown. Other examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/−DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2), the entire teachings of which are incorporated herein by reference.

Host cells are transformed with the above-described expression or cloning vectors for antibody production and cultured in conventional nutrient media modified as appropriate for inducing promoters, selecting transformants, or amplifying the genes encoding the desired sequences.

The host cells used to produce an antibody may be cultured in a variety of media. Commercially available media such as Ham's F10™ (Sigma), Minimal Essential Medium™ ((MEM), (Sigma), RPMI-1640 (Sigma), and Dulbecco's Modified Eagle's Medium™ ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. No. Re. 30,985 may be used as culture media for the host cells, the entire teachings of which are incorporated herein by reference. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as gentamycin drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

Host cells can also be used to produce portions of intact antibodies, such as Fab fragments or scFv molecules. It is understood that variations on the above procedure are within the scope of the present invention. For example, in certain embodiments it may be desirable to transfect a host cell with DNA encoding either the light chain or the heavy chain (but not both) of an antibody of this invention. Recombinant DNA technology may also be used to remove some or all of the DNA encoding either or both of the light and heavy chains that is not necessary for antigen binding. The molecules expressed from such truncated DNA molecules are also encompassed by the antibodies of the invention. In addition, bifunctional antibodies may be produced in which one heavy and one light chain are an antibody of the invention and the other heavy and light chain are specific for an antigen other than the original antigen by crosslinking an antibody of the invention to a second antibody by standard chemical crosslinking methods.

In a suitable system for recombinant expression of an antibody, or antigen-binding portion thereof, of the invention, a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells and recover the antibody from the culture medium.

When using recombinant techniques, the antibody can be produced intracellularly, in the periplasmic space, or directly secreted into the medium. In one aspect, if the antibody is produced intracellularly, as a first step, the particulate debris, either host cells or lysed cells (e.g., resulting from homogenization), can be removed, e.g., by centrifugation or ultrafiltration. Where the antibody is secreted into the medium, supernatants from such expression systems can be first concentrated using a commercially available protein concentration filter, e.g., an Amicon™ or Millipore Pellicon™ ultrafiltration unit.

Prior to the process of the invention, procedures for purification of antibodies from cell debris initially depend on the site of expression of the antibody. Some antibodies can be secreted directly from the cell into the surrounding growth media; others are made intracellularly. For the latter antibodies, the first step of a purification process typically involves: lysis of the cell, which can be done by a variety of methods, including mechanical shear, osmotic shock, or enzymatic treatments. Such disruption releases the entire contents of the cell into the homogenate, and in addition produces subcellular fragments that are difficult to remove due to their small size. These are generally removed by differential centrifugation or by filtration. Where the antibody is secreted, supernatants from such expression systems are generally first concentrated using a commercially available protein concentration filter, e.g., an Amicon™ or Millipore Pellicon™ ultrafiltration unit. Where the antibody is secreted into the medium, the recombinant host cells can also be separated from the cell culture medium, e.g., by tangential flow filtration. Antibodies can be further recovered from the culture medium using the antibody purification methods of the invention.

### 5.4. ANTIBODY PURIFICATION

5.4.1 Antibody Purification Generally

The invention provides a method for producing a purified (or “HCP-reduced”) antibody preparation from a mixture comprising an antibody and at least one HCP. The purification process of the invention begins at the separation step when the antibody has been produced using methods described above and conventional methods in the art. Table 1 summarizes one embodiment of a purification scheme. Variations of this scheme, including, but not limited to, variations where the order of the ion exchange steps is reversed, are envisaged and are within the scope of this invention.

TABLE 1
Purification steps with their associated purpose

| Purification step | Purpose |
|---|---|
| Primary recovery | clarification of sample matrix |
| Affinity chromatography | antibody capture, host cell protein and |
| | associated impurity reduction |
| Low pH incubation | viral reduction/inactivation |
| Anion exchange | antibody capture, host cell protein and |
| chromatography | associated impurity reduction |
| Hydrophobic interaction | reduction of antibody aggregates and host cell |
| chromatography | proteins |
| Viral filtration | removal of large viruses, if present |
| ultrafiltration/diafiltration | concentration and buffer exchange |
| Final filtration | concentrate and formulate antibody |

Once a clarified solution or mixture comprising the antibody has been obtained, separation of the antibody from the other proteins produced by the cell, such as HCPs, is performed using a combination of different purification techniques, including affinity separation steps(s), ion exchange separation step(s), and hydrophobic interaction separation step(s). The separation steps separate mixtures of proteins on the basis of their binding characteristics, charge, degree of hydrophobicity, or size. In one aspect of the invention, separation is performed using chromatography, including affinity, cationic, anionic, and hydrophobic interaction. Several different chromatography resins are available for each of these techniques, allowing accurate tailoring of the purification scheme to the particular protein involved. The essence of each of the separation methods is that proteins can be caused either to traverse at different rates down a column, achieving a physical separation that increases as they pass further down the column, or to adhere selectively to the separation medium, being then differentially eluted by different solvents. In some cases, the antibody is separated from impurities when the impurities specifically adhere to the column and the antibody does not, i.e., the antibody is present in the flow through.

As noted above, accurate tailoring of a purification scheme relies on consideration of the protein to be purified. In certain embodiments, the separation steps of the instant invention are employed to separate an antibody from one or more HCPs. While the present invention is directed to protein purification generally, it can be specifically adapted to the purification of antibodies. For example, antibodies that can be successfully purified using the methods described herein include, but are not limited to, human IgA₁, IgA₂, IgD, IgE, IgG₁, IgG₂, IgG₃, IgG₄, and IgM antibodies. In certain embodiments, the purification strategies of the instant invention exclude the use of Protein A affinity chromatography, for example in the context of the purification of IgG₃ antibodies, as IgG₃ antibodies bind to Protein A inefficiently. Other factors that allow for specific tailoring of a purification scheme include, but are not limited to: the presence or absence of an Fc region (e.g., in the context of full length antibody as compared to an Fab fragment thereof) because Protein A binds to the Fc region; the particular germline sequences employed in generating to antibody of interest; and the amino acid composition of the antibody (e.g., the primary sequence of the antibody as well as the overall charge/hydrophobicity of the molecule). Antibodies sharing one or more characteristic can be purified using purification strategies tailored to take advantage of that characteristic.

5.4.2. Simulated Moving Bed Chromatography

As outlined above, antibody purification typically incorporates one or more chromatography separation steps. While such chromatographic separation steps are traditionally performed in batch mode, such batch mode separations can introduce significant inefficiencies into the purification process. For example, because the use of chromatography columns in batch mode requires the columns be loaded only to their dynamic capacity, batch mode requires the use of two to three times more resin than if the columns were to be loaded to their saturation capacity. This inefficiency can greatly increase overall costs as protein chromatography resins are often very expensive. Additionally, wash and elution processes in batch column chromatography require substantial fluid volumes, which not only increase the cost of the purification process, but also substantially increase the time needed to complete such separations.

In certain embodiments, the present invention is directed to the use of one or more simulated moving bed (SMB) chromatographic separations. In certain embodiments, such SMB separations are in addition to, or take the place of, one or more traditional batch mode separations. Because SMB chromatographic separations involve the use of columns that are loaded closer to their saturation capacity, they require smaller volumes of chromatographic resin. Furthermore, because SMB separations allow for more efficient wash and elution processes, the use of SMB separations lead to substantially reduced consumption of buffers and more time-efficient purification processes.

In certain embodiments, a SMB system will include one or more modules filed with solid phase chromatographic support. Such supports include, but are not limited to, affinity chromatographic resins, ion exchange chromatographic resins, and hydrophobic interaction chromatographic resins. In certain embodiments, a particular module can include one or a plurality of chromatographic columns, provided that the system comprises at least two chromatography columns. In certain embodiments, the various aspects of each module, as well as each module in a multi-module system, are in fluid communication with each other. In certain embodiments, such fluid communication is achieved via interconnected fluid conduits. In certain embodiments, such conduits are separated by valves or other means for permitting the introduction and/or withdrawal of fluid.

In certain embodiments of the present invention, fluid conduits interconnect the upstream and downstream ends of the SMB system to form a loop through which a fluid mixture is continuously circulated. At certain points fluid streams may be introduced and at other points effluent streams may be withdrawn. In certain embodiments, a manifold system of pipes and valves can be provided to selectively position an inlet for feed material, an inlet for elution buffer, an outlet for a disassociated component and an outlet for an unassociated (or less associated) component. In certain embodiments, each inlet and outlet point communicates with a separate module or column. For example, in certain embodiments, feed material enters the system at a designated point and is moved through the solid phase by continuous internal recirculation flow. This moving contact results in a chromatographic separation of components of the feed material. Unassociated components, which flow at a relatively fast rate, are removed from an unassociated component outlet, such as by removal of a first wash effluent stream. A buffer which disassociates an associated compound from the solid phase is added at its inlet value between the respective outlet valve positions of the associated and unassociated components.

In certain embodiments, the designated inlet and outlet valve positions are displaced downstream one position on the manifold to the next solid phase bed column. The step time is chosen such that the designation of valves is properly synchronized with the internal recirculation flow. Under these conditions the system eventually reaches a steady state with specific product characteristics appearing at predetermined intervals in sequence at each valve position. This type of system simulates valves held in a single position while the solid phase moves at a constant and continuous rate around the recirculation loop producing constant quality product at each valve. In certain embodiments, an alternative apparatus can be employed where the columns are physically moved, either manually or via a mechanical carousel, while the valve locations remain fixed.

SMB separation processes approach the character of an actual moving bed system as the number of modules and valve positions increase. In certain embodiments, the number of modules will be up to 2, 3, 4, 5, 6, 7, 8, 9, or 10, where each module comprises one or more individual chromatography columns. In particular embodiments, the SMB system will include 4 or 8 chromatography columns.

In certain embodiments, the simulated moving bed process is employed in the context of affinity chromatography. In certain embodiments the chromatographic material is capable of selectively or specifically binding to the antibody of interest. Non-limiting examples of such chromatographic material include: Protein A, Protein G, chromatographic material comprising the antigen bound by the antibody of interest, and chromatographic material comprising an Fc binding protein. In specific embodiments, the affinity chromatography step involves subjecting the primary recovery sample to a column comprising a suitable Protein A resin. Protein A resin is useful for affinity purification and isolation of a variety antibody isotypes, particularly IgG₁, IgG₂, and IgG₄. Protein A is a bacterial cell wall protein that binds to mammalian IgGs primarily through their Fc regions. In its native state, Protein A has five IgG binding domains as well as other domains of unknown function.

In certain embodiments, the simulated moving bed process is employed in the context of ion exchange chromatography. Ion exchange separation includes any method by which two substances are separated based on the difference in their respective ionic charges, and can employ either cationic exchange material or anionic exchange material.

The use of a cationic exchange material versus an anionic exchange material is based on the overall charge of the protein. Therefore, it is within the scope of this invention to employ an anionic exchange step prior to the use of a cationic exchange step, or a cationic exchange step prior to the use of an anionic exchange step. Furthermore, it is within the scope of this invention to employ only a cationic exchange step, only an anionic exchange step, or any serial combination of the two.

Ion exchange chromatography may also be used as an ion exchange separation technique. Ion exchange chromatography separates molecules based on differences between the overall charge of the molecules. For the purification of an antibody, the antibody must have a charge opposite to that of the functional group attached to the ion exchange material, e.g., resin, in order to bind. For example, antibodies, which generally have an overall positive charge in the buffer pH below its pI, will bind well to cation exchange material, which contain negatively charged functional groups.

In ion exchange chromatography, charged patches on the surface of the solute are attracted by opposite charges attached to a chromatography matrix, provided the ionic strength of the surrounding buffer is low. Elution is generally achieved by increasing the ionic strength (i.e., conductivity) of the buffer to compete with the solute for the charged sites of the ion exchange matrix. Changing the pH and thereby altering the charge of the solute is another way to achieve elution of the solute. The change in conductivity or pH may be gradual (gradient elution) or stepwise (step elution).

Anionic or cationic substituents may be attached to matrices in order to form anionic or cationic supports for chromatography. Non-limiting examples of anionic exchange substituents include diethylaminoethyl (DEAF), quaternary aminoethyl (QAE) and quaternary amine (Q) groups. Cationic substituents include carboxymethyl (CM), sulfoethyl (SE), sulfopropyl (SP), phosphate (P) and sulfonate (S). Cellulose ion exchange resins such as DE23™, DE32™, DE52™, CM-23™, CM-32™, and CM-52™ are available from Whatman Ltd. Maidstone, Kent, U.K. SEPHADEX®-based and -locross-linked ion exchangers are also known. For example, DEAE-, QAE-, CM-, and SP-SEPHADEX® and DEAE-, Q-, CM- and S-SEPHAROSE® and SEPHAROSE® Fast Flow are all available from Pharmacia AB. Further, both DEAE and CM derivitized ethylene glycol-methacrylate copolymer such as TOYOPEARL™ DEAE-650S or M and TOYOPEARL™ CM-650S or M are available from Toso Haas Co., Philadelphia, Pa.

An ion exchange step facilitates the capture of the antibody of interest while reducing impurities such as HCPs. In certain aspects, the ion exchange column is a cation exchange column. For example, but not by way of limitation, a suitable resin for such a cation exchange column is CM HyperDF™ resin. These resins are available from commercial sources such as Pall Corporation. This cation exchange procedure can be carried out at or around room temperature.

In certain embodiments, the simulated moving bed process is employed in the context of hydrophobic interaction chromatography (“HIC”). Whereas ion exchange chromatography relies on the charges of the antibodies to isolate them, hydrophobic interaction chromatography uses the hydrophobic properties of the antibodies. Hydrophobic groups on the antibody interact with hydrophobic groups on the column. The more hydrophobic a protein is the stronger it will interact with the column. Thus, the HIC step removes host cell derived impurities (e.g., DNA and other high and low molecular weight product-related species).

Hydrophobic interactions are strongest at high ionic strength, therefore, this form of separation is conveniently performed following salt precipitations or ion exchange procedures. Adsorption of the antibody to a HIC column is favored by high salt concentrations, but the actual concentrations can vary over a wide range depending on the nature of the antibody and the particular HIC ligand chosen. Various ions can be arranged in a so-called soluphobic series depending on whether they promote hydrophobic interactions (salting-out effects) or disrupt the structure of water (chaotropic effect) and lead to the weakening of the hydrophobic interaction. Cations are ranked in terms of increasing salting out effect as Ba++; Ca++; Mg++; Li+; Cs+; Na+; K+; Rb+; NH4+, while anions may be ranked in terms of increasing chaotropic effect as P0−−; S04−−; CH3CO3−; Cl−; Br−; NO3−; ClO4−; I−; SCN−.

In general, Na, K or NH₄ sulfates effectively promote ligand-protein interaction in HIC. Salts may be formulated that influence the strength of the interaction as given by the following relationship: (NH₄)₂SO₄>Na₂SO₄>NaCl>NH₄Cl>NaBr>NaSCN. In general, salt concentrations of between about 0.75 and about 2 M ammonium sulfate or between about 1 and 4 M NaCl are useful.

HIC columns normally comprise a base matrix (e.g., cross-linked agarose or synthetic copolymer material) to which hydrophobic ligands (e.g., alkyl or aryl groups) are coupled. A suitable HIC column comprises an agarose resin substituted with phenyl groups (e.g., a Phenyl Sepharose™ column). Many HIC columns are available commercially. Examples include, but are not limited to, Phenyl Sepharose™ 6 Fast Flow column with low or high substitution (Pharmacia LKB Biotechnology, AB, Sweden); Phenyl Sepharose™ High Performance column (Pharmacia LKB Biotechnology, AB, Sweden); Octyl Sepharose™ High Performance column (Pharmacia LKB Biotechnology, AB, Sweden); Fractogel™ EMD Propyl or Fractogel™ EMD Phenyl columns (E. Merck, Germany); Macro-Prep™ Methyl or Macro-Prep™ t-Butyl Supports (Bio-Rad, California); WP HI-Propyl (C3)™ column (J. T. Baker, New Jersey); and Toyopearl™ ether, phenyl or butyl columns (TosoHaas, Pa.).

### 5.5. EXEMPLARY PURIFICATION STRATEGIES

In certain embodiments, the SMB separation process will employ a series of Protein A columns. In a particular, non-limiting example, the SMB separation process will employ four Protein A columns. In specific embodiments, the four Protein A columns have a diameter of 1.6 cm and a height of 5 cm and are packed with MabSelect Protein A resin. In alternative embodiments additional columns can be employed, e.g., 5, 6, 7, 8, 9, or 10 columns, and the columns can be of substantially larger diameter and height, resulting in packed column volumes of up to about 2, up to about 3, up to about 4, up to about 5, up to about 6, up to about 7, up to about 8, up to about 9, up to about 10, up to about 11, up to about 12, up to about 13, up to about 14, up to about 15, up to about 16, up to about 17, up to about 18, up to about 19, up to about 20, up to about 25, up to about 30, up to about 40, up to about 50, up to about 60, up to about 70, up to about 80, up to about 90, up to about 100, or more liters.

In certain embodiments, each Protein A column employed in a particular SMB separation scheme can be equilibrated with a suitable buffer prior to sample loading. A non-limiting example of a suitable buffer is a Tris buffer, pH of about 7.2. A non-limiting example of suitable equilibration conditions is 350 mM Tris, pH of about 7.2. Following this equilibration, the sample can be loaded onto the column. Following the loading of the column, the column can be washed one or multiple times using, e.g., the equilibrating buffer. Other washes, including washes employing different buffers, can be employed prior to eluting the column. For example, the column can be washed using one or more column volumes of 25 mM Tris at pH of about 7.2. This wash can optionally be followed by one or more washes using the equilibrating buffer. The Protein A column can then be eluted using an appropriate elution buffer. A non-limiting example of a suitable elution buffer is an acetic acid/NaCl buffer, pH of about 3.5. Suitable conditions are, e.g., 0.1 M acetic acid, pH of about 3.5. The eluate can be monitored using techniques well known to those skilled in the art. For example, the absorbance at OD₂₈₀ can be followed. Column eluate can be collected starting with an initial deflection of about 0.5 AU to a reading of about 0.5 AU at the trailing edge of the elution peak. The elution fraction(s) of interest can then be prepared for further processing. For example, the collected sample can be titrated to a pH of about 5.0 using Tris (e.g., 1.0 M) at a pH of about 10. Optionally, this titrated sample can be filtered and further processed.

In certain embodiments, sample loading is calculated to result in a particular target residence time. In specific embodiments, sample loading is calculated to result in a target residence time ranging from about 0.5 to about 12 minutes, in certain embodiments it is selected from the group consisting of up to about 0.5, up to about 1, up to about 2, up to about 3, up to about 4, up to about 5, up to about 6, up to about 7, up to about 8, up to about 9, or up to about 10 minutes. In certain embodiments, the target resident time is 3 minutes. In certain embodiments sample loading is also calculated to result in up to about 50, up to about 60, up to about 70, up to about 80, up to about 90, or up to about 100% of the saturated binding capacity of the column.

In certain embodiments, the SMB separation process involves a particular program for the introduction of equilibration, loading, washing, elution, regeneration, and storage buffers. In certain embodiments, the program will consist of three parts: 1st run, 2nd to (n−1)th run, and the last run. A non-limiting example of such a program is as follows:

Procedure (1^{st} run)

| | | Volume | Flow rate | Time | Sample |
|---|---|---|---|---|---|
| Step | (CV) | (ml) | (ml/min) | (min) | Collection |
| Equilibration | 5.0 | 50.3 | 10.1 | 5.0 | |
| Load 1 | 32.1 | 322.6 | 10.1 | 32.1 | |
| Wash 1 | 5.0 | 50.3 | 10.1 | 5.0 | |
| Wash 2 | 5.0 | 50.3 | 10.1 | 5.0 | |
| Elution | 5.0 | 50.3 | 10.1 | 5.0 | Collect > 100mAU |
| | | | | | in F5 |
| Regeneration | 5.0 | 50.3 | 10.1 | 5.0 | |
| Storage | 5.0 | 50.3 | 10.1 | 5.0 | |
| | Solution | Volume | Flow rate | Time | Sample |
| Step | (CV) | (ml) | (ml/min) | (min) | Collection |

Procedures (2^{nd} run to (n − 1)^{th} run)

| | | | | | |
|---|---|---|---|---|---|
| Equilibration | 5.0 | 50.3 | 10.1 | 5.0 | |
| Load 2 | 26.5 | 266.4 | 10.1 | 26.5 | |
| Wash 1 | 5.0 | 50.3 | 10.1 | 5.0 | |
| Wash 2 | 5.0 | 50.3 | 10.1 | 5.0 | |
| Elution | 5.0 | 50.3 | 10.1 | 5.0 | Collect > 100mAU |
| | | | | | in F5 |
| Regeneration | 5.0 | 50.3 | 10.1 | 5.0 | |
| Storage | 5.0 | 50.3 | 10.1 | 5.0 | |

Procedure (last run)

| | | | | | |
|---|---|---|---|---|---|
| Equilibration | 5.0 | 50.3 | 10.1 | 5.0 | |
| Wash 1 | 5.0 | 50.3 | 10.1 | 5.0 | |
| Wash 2 | 5.0 | 50.3 | 10.1 | 5.0 | |
| Elution | 5.0 | 50.3 | 10.1 | 5.0 | Collect > 100mAU |
| | | | | | in F5 |
| Regeneration | 5.0 | 50.3 | 10.1 | 5.0 | |
| Storage | 5.0 | 50.3 | 10.1 | 5.0 | |

In certain embodiments, the first wash step employs an identical buffer as the equilibration buffer. In certain embodiments, the first wash step can be integrated into the sample loading step. In certain embodiments the wash, elution, and regeneration steps can be calculated and programmed in order to keep the loading time to be about 50% of the run.

### 5.5. RAMAN SPECTROSCOPY

Raman spectroscopy is based on the principle that monochromatic incident radiation on materials will be reflected, absorbed or scattered in a specific manner, which is dependent upon the particular molecule or protein which receives the radiation. While a majority of the energy is scattered at the same wavelength (Rayleigh scatter), a small amount (e.g., 10⁻⁷) of radiation is scattered at some different wavelength (Stokes and Antistokes scatter). This scatter is associated with rotational, vibrational and electronic level transitions. The change in wavelength of the scattered photon provides chemical and structural information.

In certain embodiments, Raman spectroscopy can be performed on multi-component mixtures, such as those employed in the context of the SMB techniques described herein, to provide a highly specific “fingerprint” of the components. The spectral fingerprint resulting from a Raman spectroscopy analysis of a mixture will be the superposition of each individual component. The relative intensities of the bands correlate with the relative concentrations of the particular components. Accordingly, in certain embodiments, Raman spectroscopy can be used to qualitatively and quantitatively characterize a mixture of components. Thus, in certain of such embodiments, Raman spectroscopy can be employed to monitor and/or determine the composition of one or more of the multi-component mixtures involved in the production of the HCP-reduced target protein preparations of the present invention.

Raman spectroscopy can be used to characterize most samples, including solids, liquids, slurries, gels, films, powders and some gases, with a very short signal acquisition time. Generally, samples can be taken directly from the bioprocess at issue, without the need for special preparation techniques. Also, incident and scattered light can be transmitted over long distances allowing remote monitoring. Furthermore, since water provides only a weak Raman scatter, aqueous samples can be characterized without significant interference from the water.

The applicable processes and compositions described herein can be analyzed based on commercially available Raman spectroscopy analyzers. For example, a RamanRX2™ analyzer, or other analyzers commercially available from Kaiser Optical Systems, Inc. (Ann Arbor, Mich.) can be employed. Alternatively, Raman analyzers commercially available from, for example, PerkinElmer (Waltham, Mass.), Renishaw (Gloucestershire, UK) and Princeton Instruments (Trenton, N.J.). Technical details and operating parameters for the commercially available Raman spectroscopy analyzers can be obtained from the respective vendors.

Suitable exposure times, sample sizes and sampling frequencies can be determined based on, for example, the Raman spectroscopy analyzer and the process for which it is employed (e.g., in processes providing real-time monitoring of UF/DF bioprocess operations). Similarly, proper probe placement can also be determined based on the analyzer and process for which the analyzer is employed. For example, the sample size for the immersion probe to provide an adequate signal can be less than 20 mL, or less than 10 mL (e.g., 8 mL or less). The exposure time to provide an adequate signal can be less than 2 minutes, or less than 1 minute (e.g., 30 seconds).

For components for which quantization is desired, and that exist at more than one pH dependent ionization forms (e.g., histidine), Raman spectroscopic calibrations can be conducted at varying concentrations, and/or at various pH's to predict the concentration over a given pH range, such that measurement of the component (e.g., histidine) is not pH-dependent. For example, calibration models for histidine in different pH-dependent forms can be used to measure and quantify histidine in various ionized forms such that solution properties can be ascertained. Signal processing can be performed, which can include an intensity correction (e.g., standard normal variate (SNV)) and/or baseline correction (e.g., a first derivative).

Exposure times can be determined by measuring CCD saturation of representative test solutions and ensuring that they are within the acceptable instrument range (e.g., 40-80%).

In some embodiments, pH control or pH range modeling is employed for particular components (e.g., buffers such as histidine). In some embodiments, incident light is minimized, which can be achieved, for example, by use of a cover to block ambient light sources from interfering with the spectroscopy (e.g., aluminum foil).

In certain embodiments, in which, for example, a protein (such as an antibody) is concentrated with non-charged species, the protein occupies a significant volume of the solution, excluding a significant amount of solute. This results in an net decrease in the concentration of the non-charged species. This effect is referred to as “Volume exclusion,” which is proportional to the protein concentration.

In certain embodiments, such as those embodiments involving assays of charged components, a Donnan Effect occurs because at higher concentrations protein charge becomes a significant contribution to the overall charged species in solution. Since an equilibrium is expected to be established on either side of the membrane, the electroneutrality requirement results in a net decrease in positively charged species (e.g., buffer species) on the retentate side of the membrane. This phenomenon is called the Donnan effect.

According to certain embodiments of the present application, a RamanRX2™ analyzer is employed. This analyzer, as well as other commercially available Raman analyzers, provides the capability of monitoring up to four channels with simultaneous full-spectral coverage. In certain embodiments, standard NIR laser excitation is employed to maximize sample compatibility. Programmable sequential monitoring formats can be employed, for example, by the RamanRX2™ analyzer, and the apparatus is compatible with process optics, enabling one analyzer type to be employed from the discovery phase to the production phase. A portable enclosure and fiber optic sampling interface allows the analyzer to be used in multiple locations.

In certain embodiments of the presently disclosed subject matter, at least one multi-component mixture standard containing pre-determined amounts of known components (i.e., multi-component mixture standards) are characterized by Raman spectroscopy in order to obtain a model for use with mixtures with unknown components and/or unknown concentrations of known or unknown components (e.g., a calibration curve). Preferably, a series of multi-component mixture standards with pre-determined amounts of known components are characterized via Raman spectroscopy for purposes of obtaining a model.

Methodologies for obtaining a model for use with mixtures with unknown components and/or unknown concentrations of known or unknown components can be determined by persons of ordinary skill in the art. For example, a Partial Least Squares Regression Analysis based on the principal components that are expected to be present in multi-component test mixtures. Also, software programs available from Raman spectroscopy vendors can be employed to design multi-component mixture standards, which in turn can be used to develop the model for use with the multi-component test mixtures.

It is understood that reference to “providing a multi-component mixture standard with pre-determined amounts of known components” and “performing a Raman Spectroscopy analysis on the multi-component mixture standard,” and more generally, developing a model to characterize multi-component mixtures with unknown components or unknown concentrations of components includes both parallel analysis (i.e., data obtained “on-site”), as well as reference to previously obtained or previously recorded results (e.g., Raman spectra fingerprints) for multi-component mixture standards, i.e., multi-component mixtures with known components with known concentrations. For example, reference to Raman spectra results obtained from vendor product literature in encompassed by “providing a multi-component mixture standard with pre-determined amounts of known components” and “performing a Raman Spectroscopy analysis on the multi-component mixture standard.

Certain embodiments of the present application employ Raman spectroscopy techniques to characterize components (e.g., multi-component mixtures) used in bioprocess operations, including, but not limited to, SMB operations. For example, in certain embodiments, Raman spectroscopy can be used to characterize formulations that are intended to be combined with a biologically active agent (e.g., a monoclonal antibody) in the context of an SMB separation. These formulations, sometimes referred to as “formulation buffers” are typically multi-component mixtures that determine excipient levels in biologics. For example, such formulations generally include one or more of the following: a pH buffer (e.g., a citrate, Tris, acetate, or histidine compound), a surfactant (e.g., polysorbate 80), a sugar or sugar alcohol (e.g., mannitol) and/or an amino acid (e.g., L-arginine or methionine). Errors in formulation buffers often result in rejected batches, which in turn result in significant loses. Use of the techniques disclosed herein can reduce or eliminate such inefficiencies.

In certain embodiments, Raman spectroscopy techniques can be used to identify protein aggregations, such as, but not limited to, those that can form during SMB operations. For example, but not by way of limitation, the Raman spectroscopy techniques of the present invention can, in certain embodiments, identify aggregations of protein Drug Substance and Drug Product samples, including, but not limited to, antibody Drug Substance samples and antibody Drug Product samples.

In certain embodiments, Raman spectroscopy can be used to test and characterize formulations present in filtration operations (e.g., ultrafiltration/diafiltration processes), such as filtration operations in which a biologically active agent, such as a monoclonal antibody is purified, including, but not limited to operations performed in conjunction with an SMB operation. For example, but not by way of limitation, the Raman spectroscopy techniques of the present invention can be used to obtain samples obtained on-line or off-line to ascertain both the identity and quantity of the components present in a single reading. In certain embodiments, protein concentrations can be determined in addition to excipient concentrations. In certain of such embodiments, protein concentrations in the range of 0 to 150 mg/ml can be analyzed.

In certain embodiments, Raman spectroscopy can be used to monitor, verify, test and hence control bioprocess operations, such as, but not limited to, those performed in conjunction with SMB operations. The unit operations that are used with bioprocess operations, e.g., chromatography, filtration, pH changes, composition changes by addition of components or dilution of solutions, all result in mixtures composed of organic or inorganic components and biological molecules. Accordingly, measuring rapidly and accurately the composition of intermediates, for example, by employing Raman spectroscopy, provides opportunities to improve and maintain consistency and quality of the operations as well as the biological product.

In certain embodiments, the measurement of the composition of individual components in a mixture by Raman spectroscopy allows for accurate preparation of such mixtures, with and without the presence of the biologic molecule. For example, in certain embodiments, such a measurement will be useful in preparation of buffer solutions used extensively in bioprocess operations with benefits of improving consistency of the preparation or providing near real time preparation of the buffer solutions. In certain embodiments, this will eliminate the need for elaborate equipment for preparation, holding and delivery of buffer solutions. In certain embodiments, the use of Raman spectroscopy allows for the testing and release of buffer solutions can be provided in which potential errors in the buffer formulations (e.g., chemical component concentrations, wrong chemicals, etc.) are detected in real-time with simple instrumentation. Formulations that can be tested include, but are not limited to, protein-free three-component formulations (buffer+sugar+amino acid), protein and sugar formulations, protein and surfactant formulations, and protein and buffer formulations.

In certain embodiments, accurate measurement of solution composition allows for adjustment of biological solutions so that the right target composition of additives (anion, cation, hydrophobic, solvents, etc.) can be achieved. Currently such measurements are tedious and require sophisticated analytical methods that are not amenable to implementation to real time use. The use of Raman spectroscopy allows for measurements that provide a very high degree of assurance with documentation, which is an expectation in regulated industries.

In certain embodiments, the techniques of the instant invention allow for the ability to monitor and control protein—protein reactions, protein—small molecule reactions, and/or protein modifications that are achieved by chemical, physical or biological means. In certain of such embodiments, the unique biochemical signature of the reactant (biologic in its original state) and the product (biologic in its final state), as well as other reactants/catalysts that are either chemical or biological in nature are monitored using Raman spectroscopy. Monitoring the reactant(s) and product(s) in this fashion allows for, among other things, feed back control of reaction conditions and reactant amounts. It is also possible, in certain embodiments, to design a system to remove reaction by-products and/or products continually to optimize, improve or maintain product quality or performance of such systems.

In certain embodiments, Raman spectroscopy also allows for biologic product isolation and purification in chromatography operations, including, but not limited to, SMB operations. In certain of such embodiments, the elution of product/product variants/product isoforms or impurities can be monitored and fractionation of column effluent can be performed based on desired product quality or process performance. In certain embodiments, it is also possible to apply Raman spectroscopy to the isolation/enrichment of fractions in other unit operations, such as, but not limited to, filtration and non-chromatographic separations.

In certain embodiments, Raman spectroscopy is capable of being deployed as a non-invasive tool. For example, but not by way of limitation, Raman spectroscopy measurements can be made through materials that do not interfere with the signal. This provides additional unique advantages in bioprocess operations where maintaining the integrity of the containers/vessels containing these mixtures is critical.

In certain embodiments, Raman spectroscopy can be an extremely valuable means of detecting “contamination” of a solution with other components. In certain of such embodiments, carryover of the chromatographic support, or a portion thereof, from one purification step to another is detected. In certain embodiments, such carryover includes, but is not limited to, Protein A leached from a Protein A chromatographic support. In certain of such embodiments, Raman spectroscopy data obtained from a contaminated solution is compared with the expected spectra using statistical or spectral comparison techniques and, if different, can allow for the rapid detection of errors in formulation of these solutions, before they are used in bioprocesses.

In certain embodiments, as demonstrated through an example below as a proof of concept, concentration of antibody in a mixture containing impurities from the cell culture harvest materials including host cell proteins, DNA, lipids etc can be measured quantitatively using Raman Spectroscopy. In such embodiments, the said method can be used to monitor influents and effluents from bioprocess operations containing unpurified mixtures. Examples could include, but not limited to loading and elution operations for columns, filters, and non-chromatographic separation devices (expanded bed, fluidized bed, two phase extractions etc). The example provided demonstrates that the antibody concentration from 0.1 to 1 g/L can be quantified in a matrix that comprises the unbound fraction from a protein A affinity chromatography column that was loaded with a clarified harvest solution prepared from a chemically defined media based cell culture process. If Raman spectroscopy is incorporated in-line, then such a measurement will enable direct monitoring and control of the column loading, enabling consistent and optimal loading of the columns either at a predefined binding capacity that represents either a percent of the dynamic binding capacity or static (equilibrium) capacity. One skilled in the art would recognize that such technology could apply to various other operations as mentioned above.

In certain embodiments, Raman spectroscopy can be used for quality control and/or feedback control in bioprocess purification operations (e.g., to control in-line buffer dilution for a therapeutic antibody purification process). In certain of such embodiments, Raman spectroscopy can be used for quality control and/or feedback control in processes involving protein conjugation reactions or other chemical reactions (e.g., a liquid-phase Heck reaction), as described in Anal. Chem., 77:1228-1236 (2005), hereby incorporated by reference in its entirety.

### 6. EXAMPLES

### 6.1. Case Study: mAb X

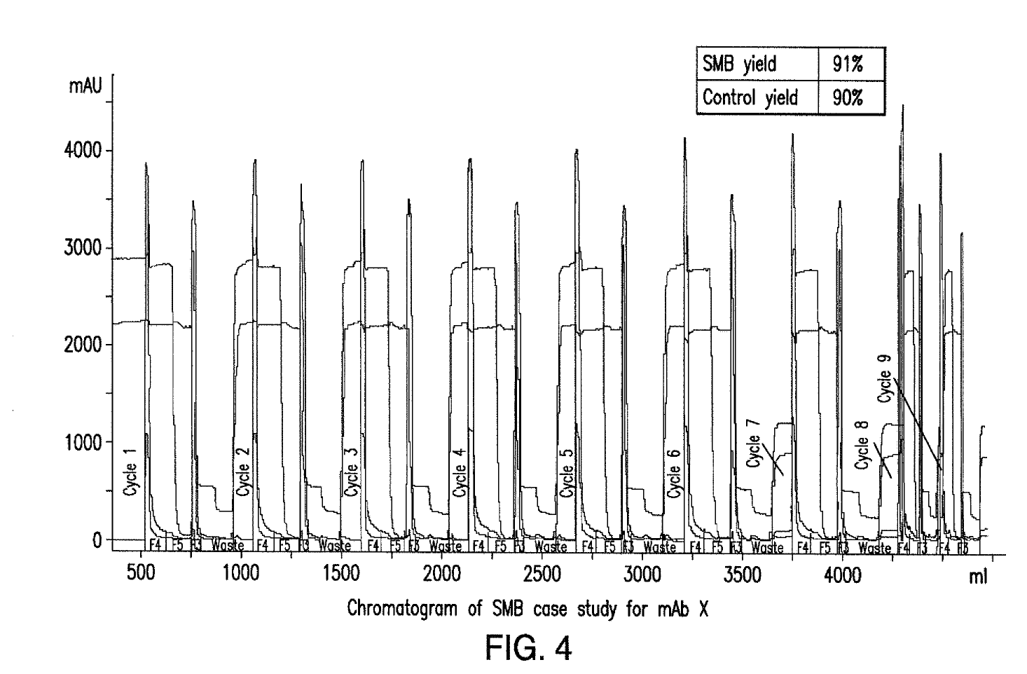

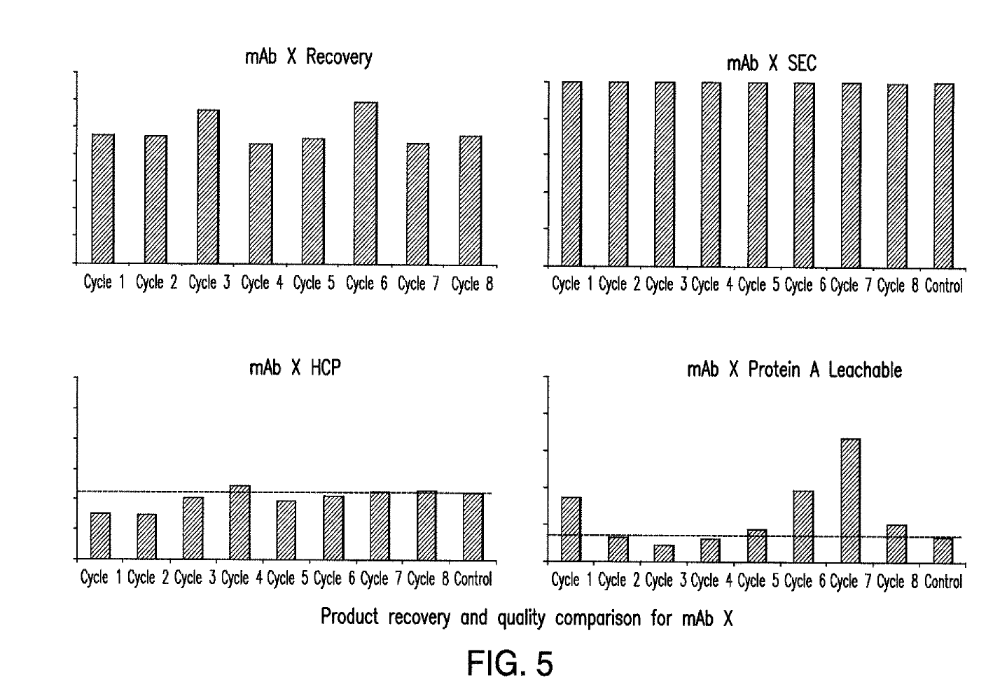

A case study was performed using an mAb X process intermediate as the feed stream, and a typical agarose-based affinity Protein A chromatography media as an affinity chromatography resin. A total of eight cycles were performed with four columns. The columns were each loaded to saturation and FIG. 4 shows the resulting chromatogram. The even-number UV peaks indicate elution and the odd-number UV peaks indicate wash 1 immediately after loading.

The following buffers were used for all SMB runs:

| Line | |
|---|---|
| positions | Buffer |
| Equili/Wash 1 | 350 mM Tris, pH 7.2 |
| Wash 2 | 25 mM Tris, pH 7.2 |
| Elution | 100 mM Na Acetate, pH 3.5 |
| Regeneration | 200 mM Acetic Acid |
| Storage | 50 mM Na Acetate, pH 5.0, 2% benzyl alcohol |

The following tables outline the SMB purification program for mAb X. There are three parts of program: 1^{st} run, 2^{nd} to (n−1)^{th} run, and the last run. The Load 2 block was calculated by the area-under-curve (AUC) of the saturated binding capacity (SBC) study (see below). Note, the following separation processes were performed at 20% less than the true SBC. The first separation process was performed at 1 minute residence time, and the second was performed at 3 minutes residence time.

Column Information:

| | | | |
|---|---|---|---|
| Resin: | MabSelect | | |
| Diameter | 1.6 cm | | |
| Height | 5 cm | | |
| Column volume: | 10.05 ml | Column area: | 2.01 cm² |
| Linear Flow: | 300 cm/hr | | |
| Flow rate: | 10.05 ml/min | Res. Time: | 1 min |
| Linear Flow: | 100 cm/hr | | |
| Flow rate: | 3.35 ml/min | Res. Time: | 3 min |

Sample Information:

| Clarified Harvest material from cell culture process. | |
|---|---|
| No dilution. | |
| 0.22 micron filtration prior to load as necessary. | |
| Titer: | 1.82 g/L |
| DBC: | 41 g/L resin |
| SBC: | 73 g/L resin |
| Target load: | 58.4 g/L resin, 20% back off SBC |

Procedure (1^{st} run)

| | | Volume | Flow rate | Time | Sample |
|---|---|---|---|---|---|
| Step | (CV) | (ml) | (ml/min) | (min) | Collection |
| Equilibration | 5.0 | 50.3 | 10.1 | 5.0 | |
| Load 1 | 32.1 | 322.6 | 10.1 | 32.1 | |
| Wash 1 | 5.0 | 50.3 | 10.1 | 5.0 | |
| Wash 2 | 5.0 | 50.3 | 10.1 | 5.0 | |
| Elution | 5.0 | 50.3 | 10.1 | 5.0 | Collect > 100mAU |
| | | | | | in F5 |
| Regeneration | 5.0 | 50.3 | 10.1 | 5.0 | |
| Storage | 5.0 | 50.3 | 10.1 | 5.0 | |
| | Solution | Volume | Flow rate | Time | Sample |
| Step | (CV) | (ml) | (ml/min) | (min) | Collection |

Procedures (2^{nd} run to (n − 1)^{th} run)

| | | | | | |
|---|---|---|---|---|---|
| Equilibration | 5.0 | 50.3 | 10.1 | 5.0 | |
| Load 2 | 26.5 | 266.4 | 10.1 | 26.5 | |
| Wash 1 | 5.0 | 50.3 | 10.1 | 5.0 | |
| Wash 2 | 5.0 | 50.3 | 10.1 | 5.0 | |
| Elution | 5.0 | 50.3 | 10.1 | 5.0 | Collect > 100mAU |
| | | | | | in F5 |
| Regeneration | 5.0 | 50.3 | 10.1 | 5.0 | |
| Storage | 5.0 | 50.3 | 10.1 | 5.0 | |

Procedure (last run)

| | | | | | |
|---|---|---|---|---|---|
| Equilibration | 5.0 | 50.3 | 10.1 | 5.0 | |
| Wash 1 | 5.0 | 50.3 | 10.1 | 5.0 | |
| Wash 2 | 5.0 | 50.3 | 10.1 | 5.0 | |
| Elution | 5.0 | 50.3 | 10.1 | 5.0 | Collect > 100mAU |
| | | | | | in F5 |
| Regeneration | 5.0 | 50.3 | 10.1 | 5.0 | |
| Storage | 5.0 | 50.3 | 10.1 | 5.0 | |

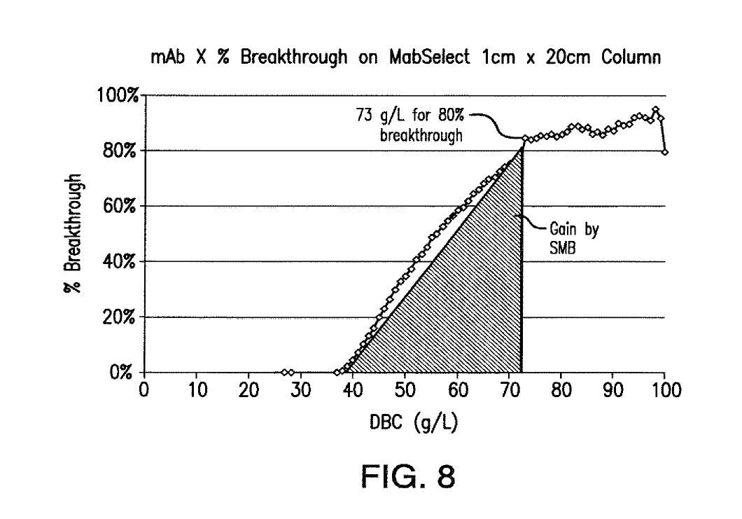

The data in FIG. 8 illustrates the saturated binding capacity (SBC) study for mAb X. The flow through of the column was analyzed by Poros A HPLC assay to determine the amount of product not bound onto the column. The area-under-curve (AUC) up to the saturated binding capacity of 73 g/L indicates the improvement achieved by SMB, vs. typical binding (“dynamic binding”) of 40 g/L. The AUC was subtracted from the saturated binding capacity to calculate the load amount for 2^{nd} cycle to the last cycle (see FIG. 8).

### 6.2. Case Study mAb Y

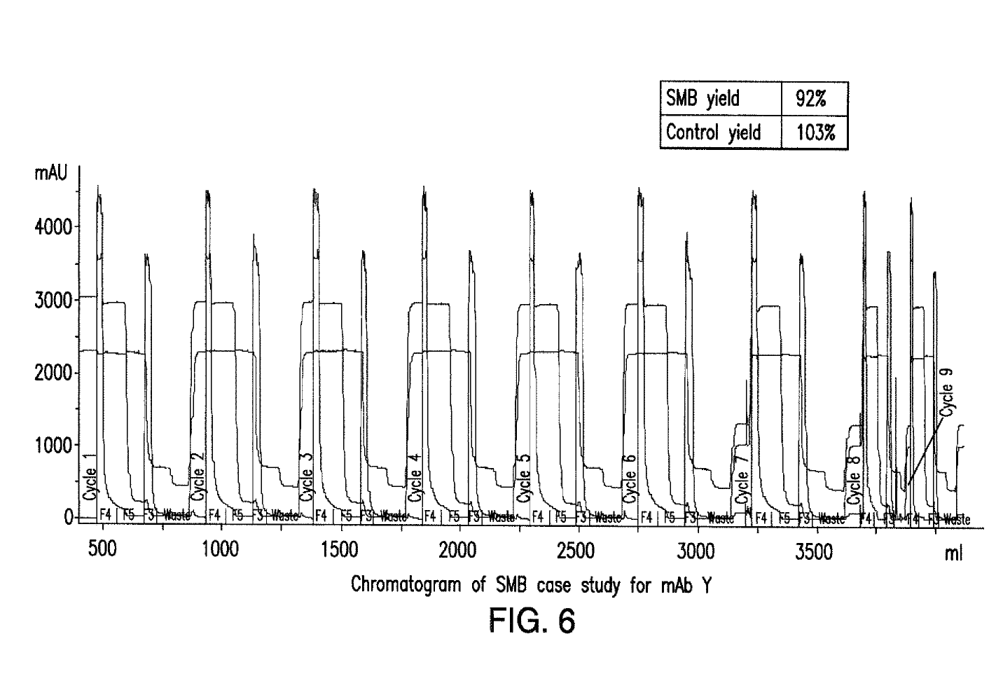

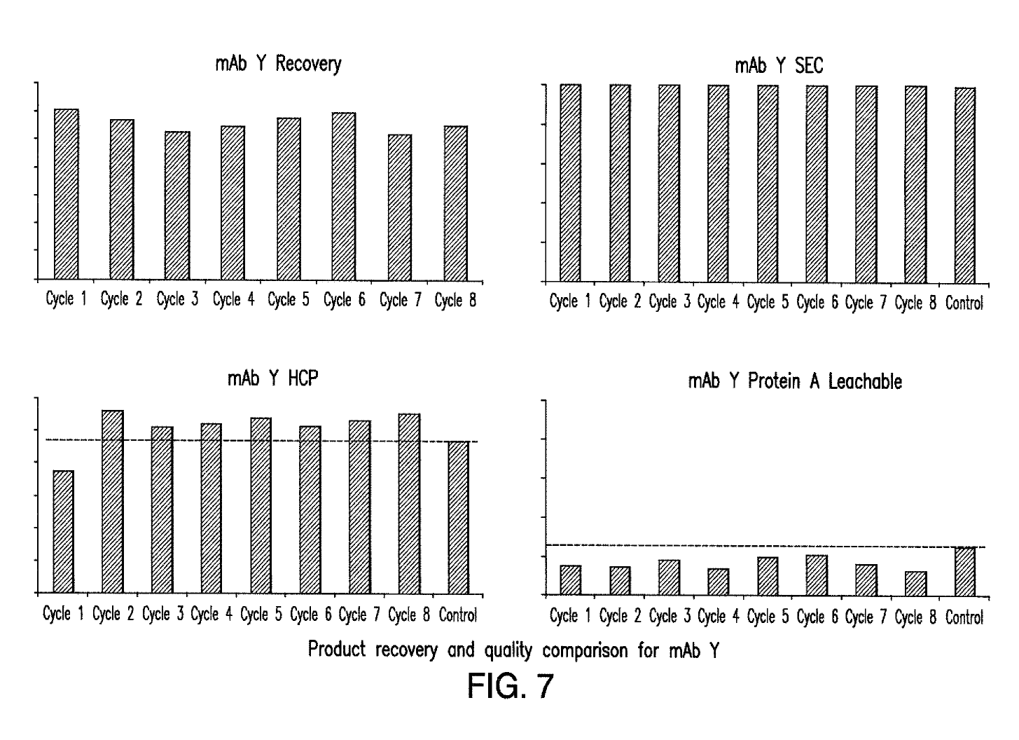

A case study was performed using an mAb Y process intermediate as the feed stream, and a typical agarose-based affinity Protein A chromatography media as an affinity chromatography resin. A total of eight cycles were performed with four columns. The columns were each loaded to saturation and FIG. 6 shows the resulting chromatogram. The even-number UV peaks indicate elution and the odd-number UV peaks indicate wash 1 immediately after loading.

These buffers were used for all SMB runs:

| Line | |
|---|---|
| positions | Buffer |
| Equili/Wash 1 | 350 mM Tris, pH 7.2 |
| Wash 2 | 25 mM Tris, pH 7.2 |
| Elution | 100 mM Na Acetate, pH 3.5 |
| Regeneration | 200 mM Acetic Acid |
| Storage | 50 mM Na Acetate, pH 5.0, 2% benzyl alcohol |

The following tables outline the SMB purification program for mAb Y. There are three parts of program: 1^{st} run, 2^{nd} to (n−1)^{th} run, and the last run. The Load 2 block was calculated by the area-under-curve (AUC) of the saturated binding capacity (SBC) study (see below). The wash 1, wash 2, and regeneration steps were calculated in order to keep the loading time to be 50% of the run and the loading step was calculated to result in a 3 minute residence time. Note, the following separation processes were performed at 20% less than the true SBC.

Column Information:

| | | | |
|---|---|---|---|
| Resin: | MabSelect | | |
| Diameter | 1.6 cm | | |
| Height | 5 cm | | |
| Column volume: | 10.05 ml | Column area: | 2.01 cm² |
| Linear Flow: | 100 cm/hr | | |
| Flow rate: | 3.35 ml/min | Res. Time: | 3 min |

Sample Information:

| Clarified Harvest material from cell culture process. | |
|---|---|
| No dilution. | |
| 0.22 micron filtration prior to load as necessary. | |
| Titer: | 2.7 g/L |
| DBC: | 52 g/L resin |
| SBC: | 86 g/L resin |
| Target load: | 68.8 g/L resin, 20% back off SBC |

Procedure (1^{st} run)

| | | Volume | Flow rate | Time | Sample |
|---|---|---|---|---|---|
| Step | (CV) | (ml) | (ml/min) | (min) | Collection |
| Equilibration | 5.0 | 50.3 | 3.4 | 15.0 | |
| Load 1 | 25.5 | 256.2 | 3.4 | 76.4 | |
| Wash 1 | 4.1 | 40.9 | 3.4 | 12.2 | |
| Wash 2 | 4.1 | 40.9 | 3.4 | 12.2 | |
| Elution | 5.0 | 50.3 | 3.4 | 15.0 | Collect > 100mAU |
| | | | | | in F5 |
| Regeneration | 4.1 | 40.9 | 3.4 | 12.2 | |
| | Solution | Volume | Flow rate | Time | Sample |
| Step | (CV) | (ml) | (ml/min) | (min) | Collection |

Procedures (2^{nd} run to (n − 1)^{th} run)

| | | | | | |
|---|---|---|---|---|---|
| Equilibration | 5.0 | 50.3 | 3.4 | 15.0 | |
| Load 2 | 22.2 | 223.2 | 3.4 | 66.6 | |
| Wash 1 | 4.1 | 40.9 | 3.4 | 12.2 | |
| Wash 2 | 4.1 | 40.9 | 3.4 | 12.2 | |
| Elution | 5.0 | 50.3 | 3.4 | 15.0 | Collect > 100mAU |
| | | | | | in F5 |
| Regeneration | 4.1 | 40.9 | 3.4 | 12.2 | |
| Storage | 5.0 | 40.9 | 3.4 | 12.2 | |

Procedure (last run)

| | | | | | |
|---|---|---|---|---|---|
| Wash 1 | 4.1 | 40.9 | 3.4 | 12.2 | |
| Wash 2 | 4.1 | 40.9 | 3.4 | 12.2 | |
| Elution | 5.0 | 50.3 | 3.4 | 15.0 | Collect > 100mAU |
| | | | | | in F5 |
| Regeneration | 4.1 | 40.9 | 3.4 | 12.2 | |
| Storage | 5.0 | 50.3 | 3.4 | 15.0 | |

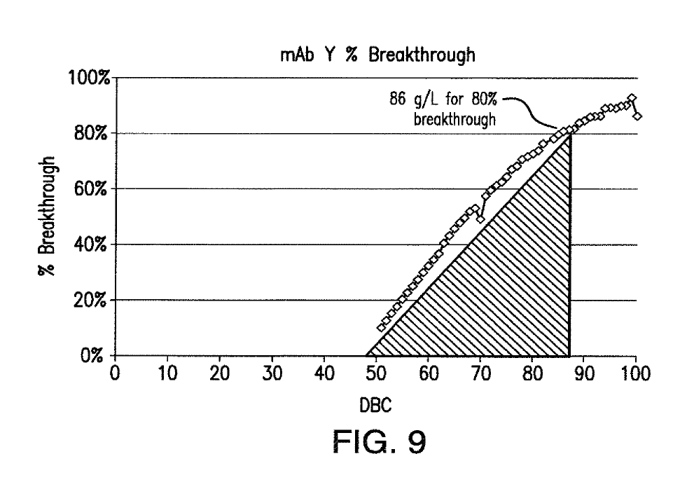

The data in FIG. 8 illustrates the saturated binding capacity (SBC) study for mAb Y. The flow through of the column was analyzed by Poros A HPLC assay to determine the amount of product not bound onto the column. The AUC up to the saturated binding capacity of 86 g/L indicates the improved binding capacity achieved by SMB vs. typical binding (“dynamic binding”) of 45 g/L. This AUC was subtracted from the saturated binding capacity to calculate the load amount for 2^{nd} cycle to the last cycle (See FIG. 9).

### 6.3. Case Study mAb X, Detection of Impurity Carryover

A second SMB separation was performed using mAb X. In this separation, mAb X was present in chemically-defined media. A total of eight cycles were performed with four columns. The columns were each loaded to saturation. The following buffers were used for all SMB runs:

| Line | |
|---|---|
| positions | Buffer |
| Equili/Wash 1 | 350 mM Tris, pH 7.2 |
| Wash 2 | 25 mM Tris, pH 7.2 |
| Elution | 100 mM Na Acetate, pH 3.5 |
| Regeneration | 200 mM Acetic Acid |
| Storage | 50 mM Na Acetate, pH 5.0, 2% benzyl alcohol |

The following table outlines the SMB purification program for mAb X. There are three parts of program: 1^{st} run, 2^{nd} to (n−1)^{th} run, and the last run. The Load 2 block was calculated by the area-under-curve (AUC) of the saturated binding capacity (SBC) study. Note, the following separation processes were performed at 20% less than the true SBC. The first separation process was performed at 1 minute residence time, and the second was performed at 3 minutes residence time.

Column Information:

| | | | |
|---|---|---|---|
| Resin: | MabSelect | | |
| Diameter | 1.6 cm | | |
| Height | 5 cm | | |
| Column volume: | 10.05 ml | Column area: | 2.01 cm² |
| Linear Flow: | 300 cm/hr | | |
| Flow rate: | 10.05 ml/min | Res. Time: | 1 min |
| Linear Flow: | 100 cm/hr | | |
| Flow rate: | 3.35 ml/min | Res. Time: | 3 min |

The overall yield of this SMB separation was 85%. Analytical assays performed included a Poros A assay to determine mAb concentration and a Protein A leachable ELISA to determine the presence of Protein A carryover after each eulate run. The latter indicated that Protein A leachable was higher for the second cycle (eluate runs 5-8) than the first cycle (eluate runs 1-4), indicating some carryover effect for this impurity.

### 6.4. Case Study mAb Z at Pilot Seale

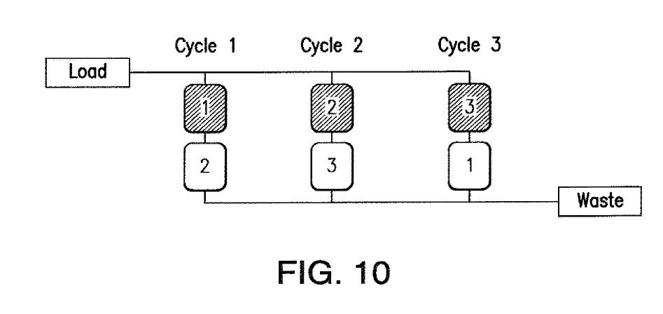

The Protein A processes described above were scaled to pilot scale and employed in the context of a mAb Z separation. Three columns (each 10 cm diameter×8 cm height, 785 mL) were packed with Protein A chromatographic support. Similar to the small scale separations outlined above, column switching was operated manually. With only three columns, the work flow can be carried out as depicted in FIG. 10.

mAb Z cell culture harvest was acidified to precipitate cells and cell debris. The precipitation of such impurities improved the effectiveness of subsequent centrifugation, and increased the capacity of the depth filters and membrane filters. Clarifying the harvest enabled sample loading onto the Protein A column with a simple wash step, and simplified cleaning procedures. An inline filter was added before the column to protect it from debris and an air trap was added before the column to protect it from air.

A simplified buffer system was used in all processing steps. The equilibration, all washes, elution buffers were composed of only two components: Tris, and Acetic Acid. With the defined amount of each component, pH can be controlled by the molarity of each component. Hence, no pH adjustment was necessary for all buffers, saving significant time in buffer preparation. The following are the buffers used in the pilot process.

| Line | |
|---|---|
| positions | Buffer |
| Equilibration | 25 mM Tris, 22 mM Acetic Acid, pH 7.2 |
| Wash 1 | 300 mM Tris, 260 mM Acetic Acid, pH 7.2 |
| Wash 2 | 25 mM Tris, 22 mM Acetic Acid, pH 7.2 |
| Elution | 25 mM Acetic Acid, 0.89 mM Tris, pH 3.5 |
| Regeneration | 0.2M sodium hydroxide |

All three columns were connected in series during cleaning, which saves the amount of cleaning agents used. The flow rate was reduced to 200 cm/hour to accommodate the increased pressure drop for three columns.

Overall yield for this process was 91%, demonstrating that mAb Z cell culture harvest can be clarified by acidification, followed by centrifugation/depth filtration, and captured by Protein A affinity chromatography, resulting a simple mAb capture process.

### 6.5 Testing of 3-Component Formulation Buffers

Formulation buffers containing predetermined mixtures of arginine, citric acid, and trehalose were prepared with a water solvent. Components were varied from 0 to 100 mM.

Raman spectra over the range of 800 to 1700 cm⁻¹ were obtained for 15 mL aliquots of each mixture using a RAMANRXN2™ Analyzer (2 spectra/mixture). The spectral filtering parameters were set to a standard normal variance (SNV) intensity normalization, a 1st derivative (gap) baseline correction with 15 point smoothing, and mean centering difference spectra with the average intensity value=0. This is considered to be a data scaling rather than a spectral filter. The spectra were collected using an immersion probe with an exposure time of 30 seconds per sample.

Principal Components Methodology was used to develop a model. A PLS (Partial Least Squares projections to latent structures) model was applied to each of the three components to determine inter-component correlations. This result is a linear model that translates spectral intensity (e.g. from 1700-800 cm⁻¹) to concentration (a×1+b×2+ . . . +z×900=concentration). The software used for the calibration results shown here was GRAMS/AI V 7.02 with the PLSplus/IQ add-in from Thermo Galactic. SIMCA P+ was used for many of the graphs and experimental model creation. The samples were cross validated by removing two samples. Data analysis was conducted so that the steps of testing for correlations and cross-validating were iterated until the inter-component correlations were below an error threshold of 2%. Accurate quantization of buffer components (e.g., within 2%) can be provided with a single reading.

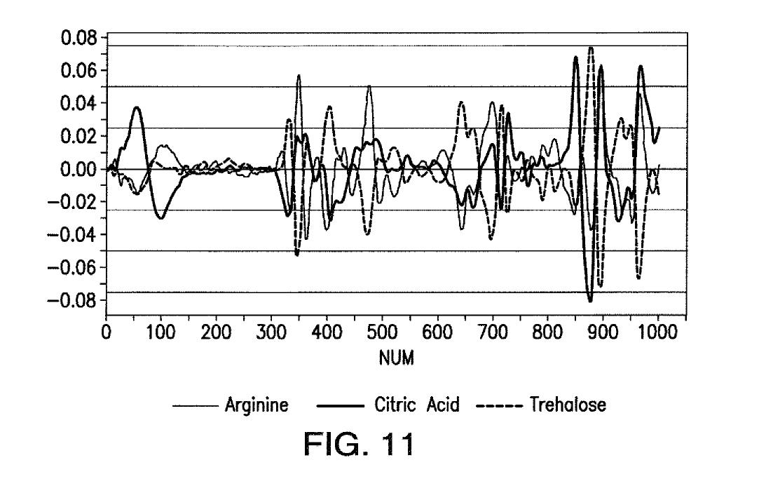

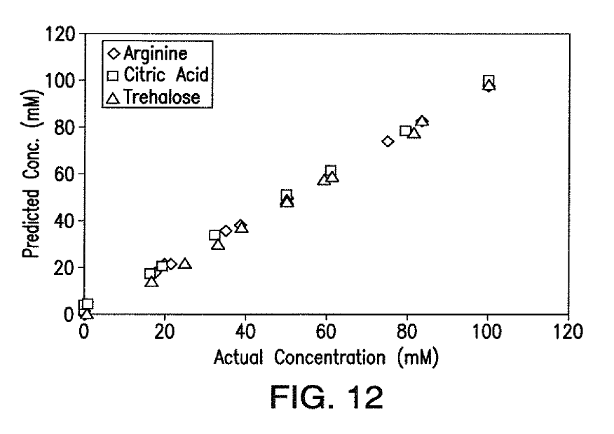

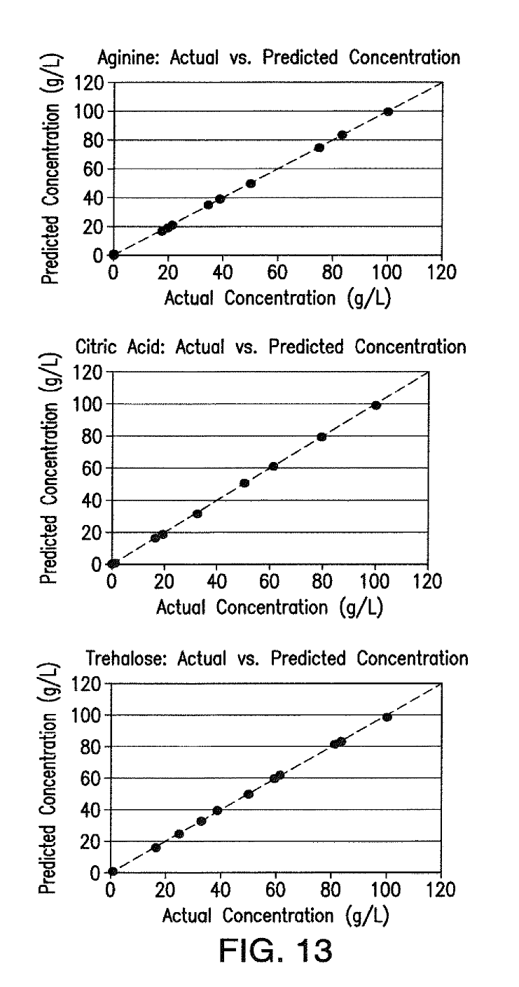

Calibration curves can be obtained using Random Mixture Design. The 3-component model developed above was used to generate predictions about spectra of random mixtures of arginine, citric acid, and trehalose (FIG. 11). These predictions were compared against the actual spectra to confirm that the model is with the pre-determined tolerance limit of +2%. The results are shown in FIGS. 12 and 13. Independent measurements were obtained of random mixtures to verify that the model can be used for making measurements.

### 6.6 Testing of 4-Component Formulation Buffers

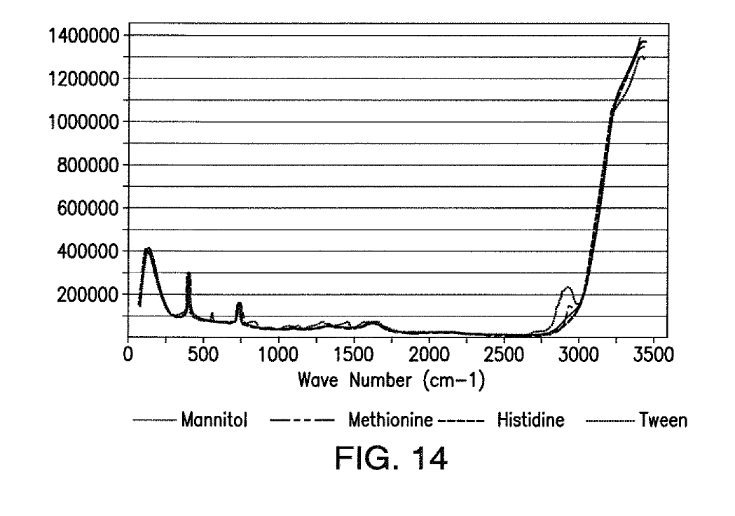

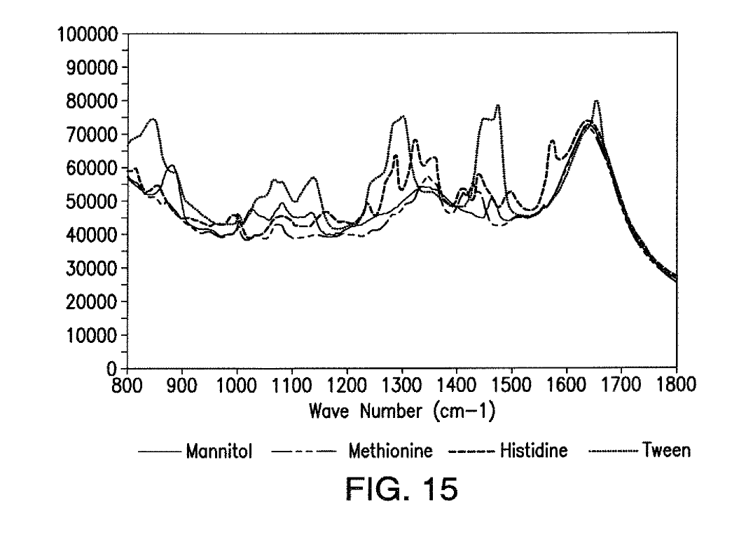

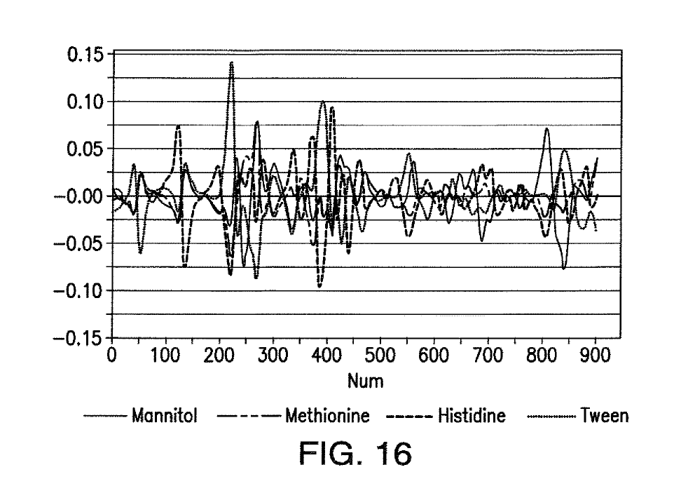

The methodology of Example 6.5 was applied to formulation buffers containing 4 components, wherein the components were mannitol, methionine, histidine, and Tween™ (polysorbate 80). The measured spectra of the predetermined mixtures are shown in FIGS. 14-16. The wave numbers range from the Far-IR region to the Mid-IR region. Due to limitations with the sapphire cover, the range from 100-800 cm⁻¹ can be disregarded in this particular example, and calibration occurs from 800-1800 cm⁻¹.

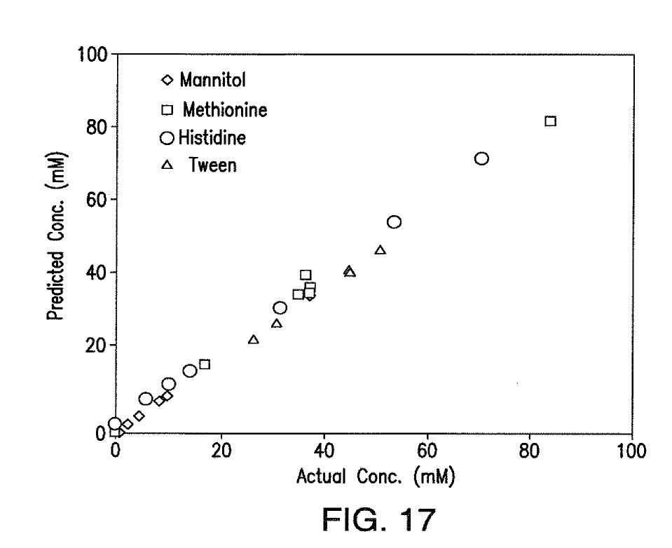

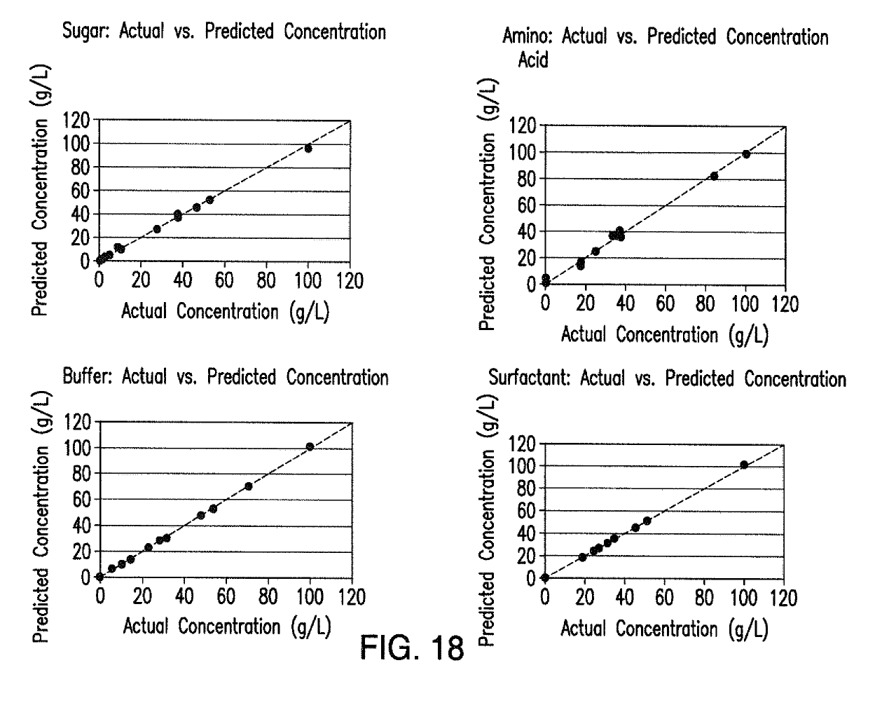

A model was obtained for a 4 component buffer system in the same manner as the 3 component model obtained in Example 6.5. The predictions based on the model obtained were compared against the actual spectra of random mixtures to confirm that the model is sufficiently accurate. The results are shown in FIGS. 17 and 18.

### 6.7 Testing of 3-Component Formulation Buffers with Protein

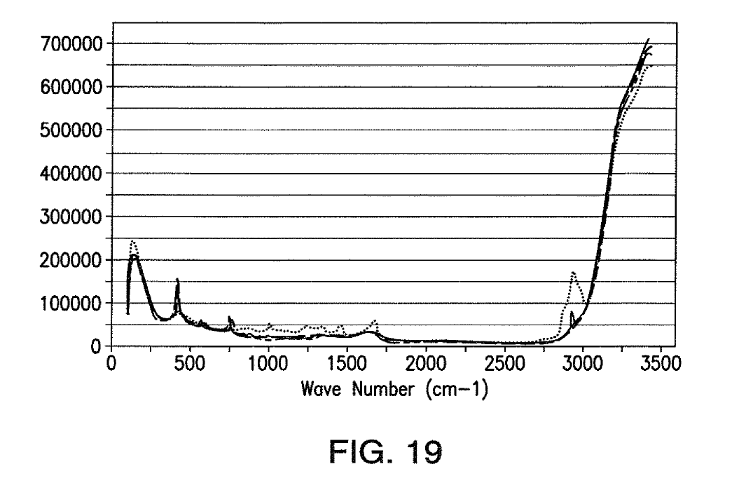

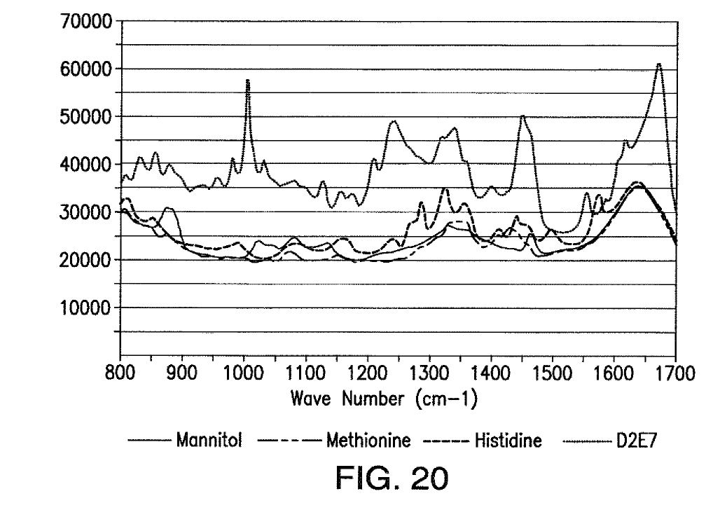

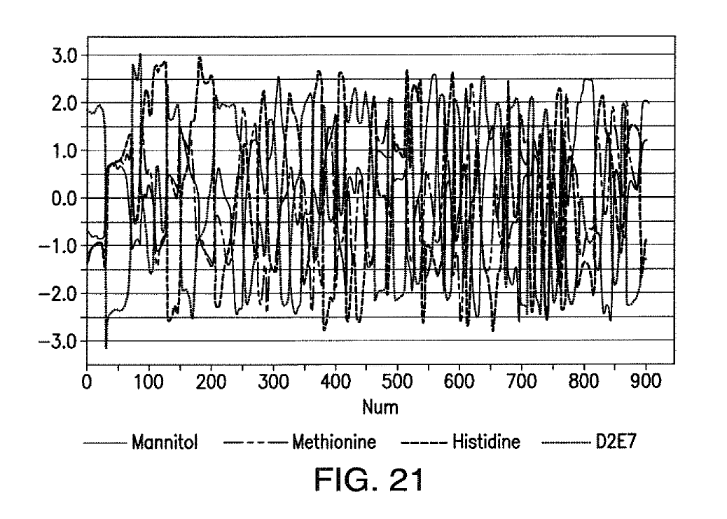

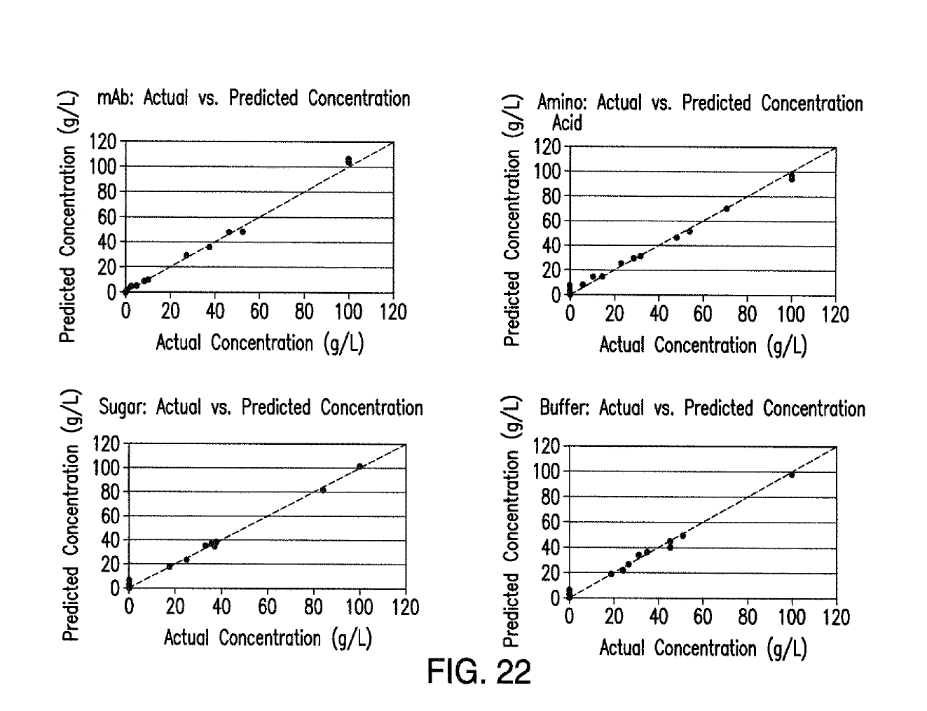

The methodology of Example 6.6 was applied to formulation buffers containing 3 components along with a protein at a concentration in the range of 0 to 100 mg/ml. The components were mannitol, methionine, histidine, and D2E7 (adalimumab). The measured spectra of the predetermined mixtures are shown in FIGS. 19-21.

A model was obtained for a 3 component buffer system with protein in the same manner as the 4 component model obtained in Example 6.6. The predictions based on the model obtained were compared against the actual spectra of random mixtures to confirm that the model is sufficiently accurate. The results are shown in FIG. 22. The coefficient of determination (R2) and standard error of cross-validation (SECV) values of the actual versus predicted spectra are show in Table 2 below.

TABLE 2
Model Fit Summary

| Component | R² | SECV (g/L) |
|---|---|---|
| Adalimumab | 0.995 | 1.96 |
| Mannitol | 0.994 | 2.35 |
| Methionine | 0.989 | 3.27 |
| Histidine | 0.992 | 2.75 |

### 6.8 Adalimumab UF/DF Process

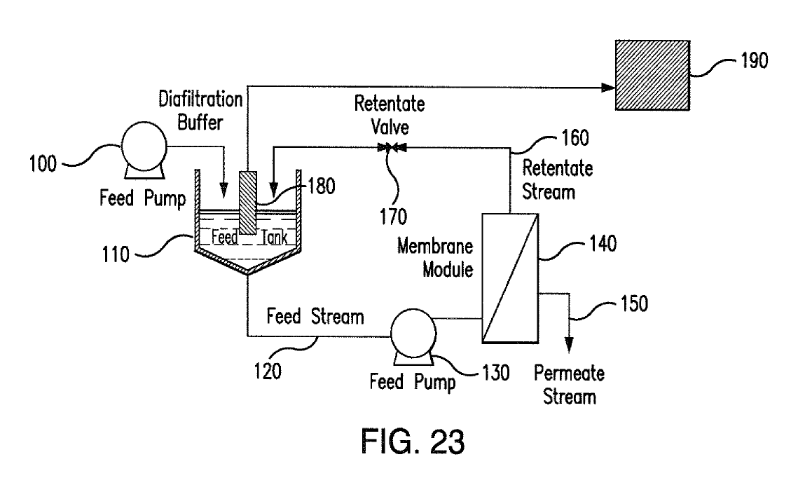

An ultrafiltration/diafiltration process (UF/DF) is established to introduce excipients into a solution of adalimumab, shown in FIG. 23. A feed pump (100) provides cross flow across the tangential flow filtration membrane, passing the adalimumab containing solution in the reservoir over the membrane. The diafiltration buffer (formulation buffer, containing Methionine, Mannitol and Histidine) is pumped into the reservoir to match the filtration rate of the membrane (liquid flowing through the permeate side of the membrane) (110). A feed stream (120) exiting the feed tank is directed by a pump (130) to a membrane module (140). A permeate stream (150) containing water, buffer components, and the like having a relatively smaller molecular size passes through the membrane module. A retentate stream (160) containing concentrated adalimumab is directed back to the feed tank, as controlled by a retentate valve (170).

A Raman probe (180), compatible with a RamanRX2™ analyzer (190) from Kaiser Opticals is placed within the feed tank to provide the ability to characterize the content of the tank periodically. The spectra obtained will be converted to component concentrations using the calibration file and hence the progress of the diafiltration process can be monitored. In addition, the changes in excipient concentrations that happen due to increase in concentration of the protein (caused by Donnan and charge exclusion effects) can be monitored and optionally controlled. Other Raman systems, besides a RamanRX2™ analyzer could also be used to characterize online samples from the ultrafiltration/diafiltration process on a regular basis as part of the Quality Control of the adalimumab purification process. For example, the results from the Raman analysis can be used to assess the completion of the diafiltration process and the final excipient concentrations.

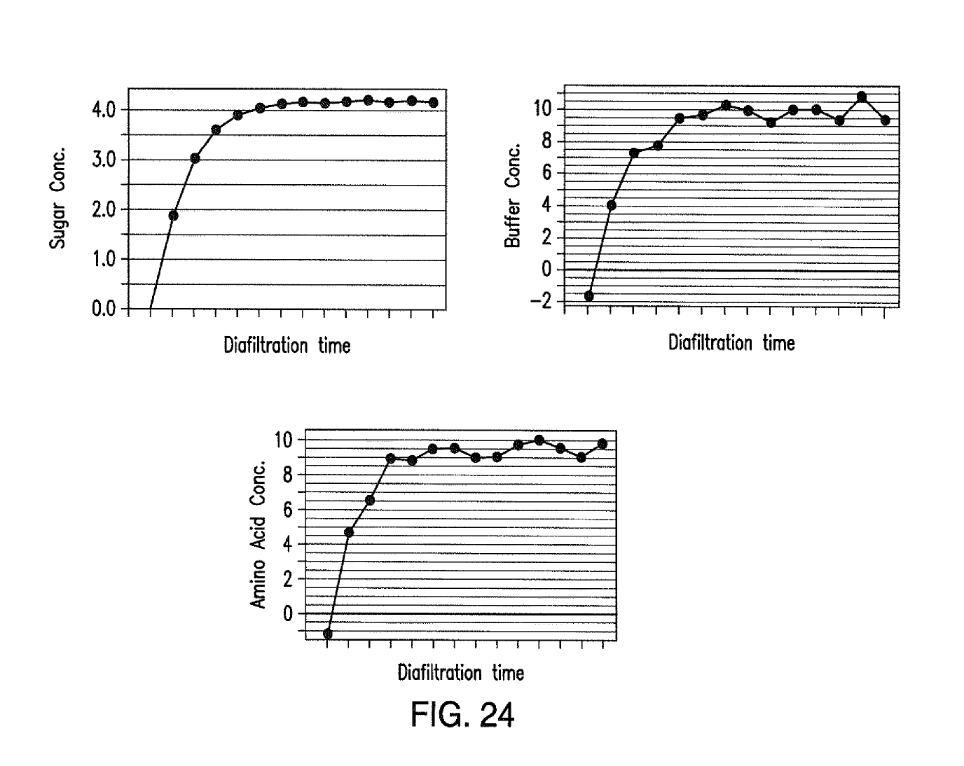

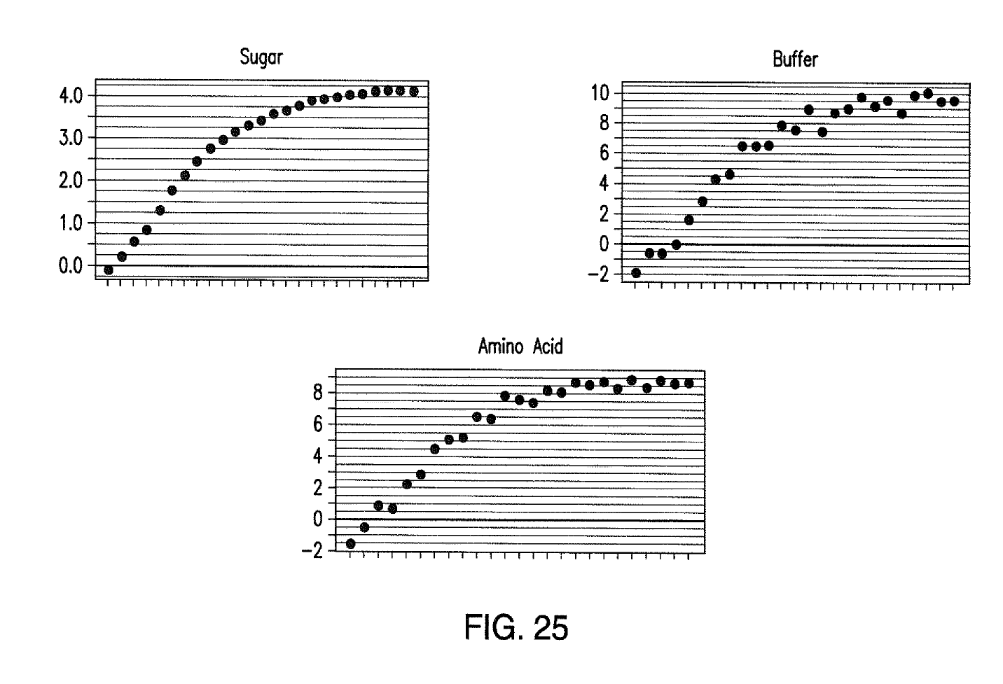

A mixture of histidine, mannitol and methionine were diafiltered across a UF/DF membrane. The raman probe was placed in the retentate reservoir. Raman Spectra were obtained at specified intervals, with each reading consisting a 30 sec exposure, repeated 10 times (10 scans). FIGS. 24-25 show the change in concentration during diafiltration. As expected the concentration of individual components increase during diafiltration reaching a plateau.

FIGS. 24-25 provide results from the on-line monitoring of the diafiltration process. In FIG. 24 sugar, buffer and amino acid concentrations are provided for various diafiltration times. As shown in FIGS. 24 and 25, amino acid is methionine, and concentration (mM) is plotted on the y-axis, sugar is mannitol, and w/v % is plotted on the y-axis, and buffer is histidine, and concentration (mM) is plotted along the y-axis. The x-axis for each of the plots in FIGS. 24-25 is retention time, in which concentrations from 0 to 81 minutes were measured and plotted along the x-axis.

Next, adalimumab at approximately 40 mg/ml present in water was diafiltered into a sugar solution over 7 diavolumes across a 5 kiloDalton UF/DF membrane (0.1 sq. in). The raman probe was placed in the retentate reservoir. Raman Spectra were obtained at specified intervals, with each reading consisting of a 30 second exposure time, repeated 10 times (10 scans). Subsequently the protein was concentrated to 140 g/L.

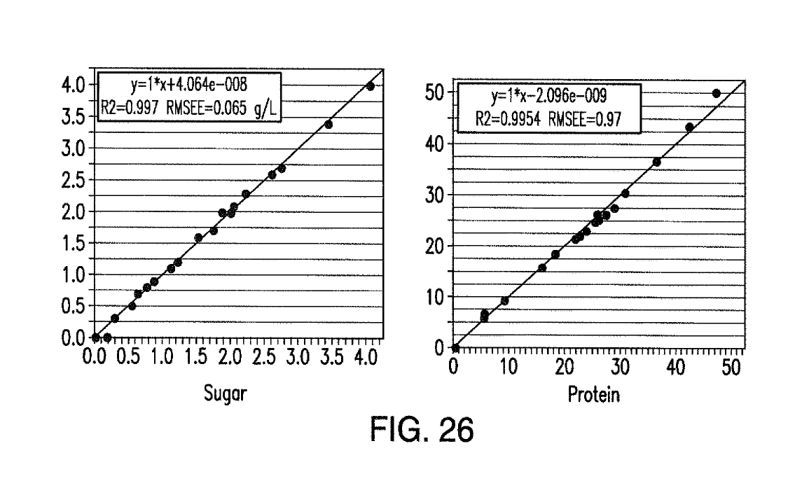

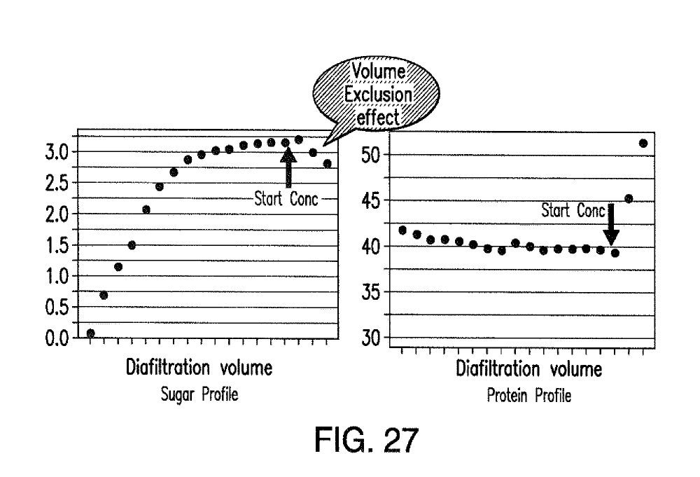

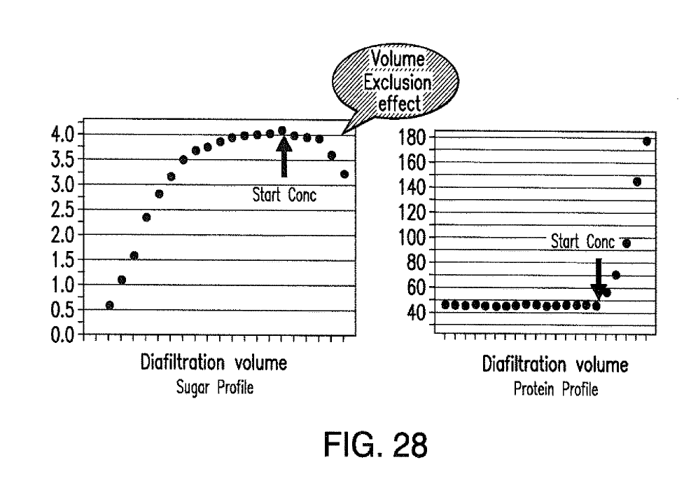

FIG. 26 provides calibration data obtained from the sugar/protein system (mannitol/adalimumab) that is employed in a UF/DF system and measured as described above. The calibration curve from FIG. 26 was used to ascertain mannitol and adalimumab concentrations in FIGS. 27 and 28. FIGS. 27 and 28 show the change in concentration during diafiltration of the sugar. The plot on the right shows the protein concentration during diafiltration and then subsequent ultrafiltration. In FIGS. 27 and 28, sugar concentration (%) is plotted versus retention volumes (from zero to 6), and adalimumab concentration (g/l) is plotted versus retention volumes (from zero to 6).

As expected the concentration of sugar increase during diafiltration reaching a plateau. The protein reaches the target concentration. In FIG. 27, a model calibrated to 50 g/L was used. FIG. 28 shows the sugar and protein concentrations calculated using calibrations obtained with 120 g/L protein and sugar mixtures.

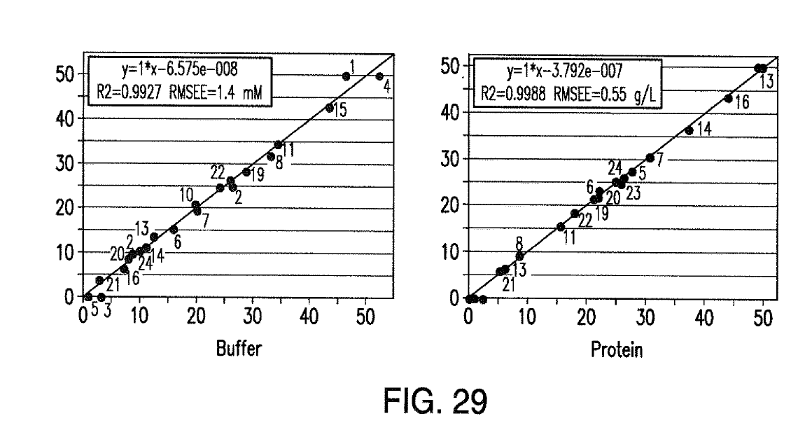

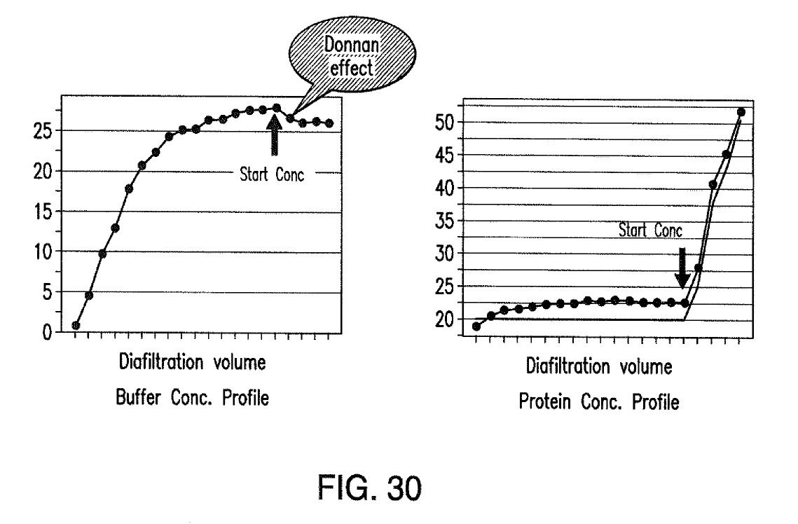

Adalimumab at approximately 20 mg/ml present in water was diafiltered into a histidine solution (50 mM) over 7 diavolumes across a 5 kiloDalton UF/DF membrane (0.1 sq. m). The raman probe was placed in the retentate reservoir. Raman Spectra were obtained at specified intervals, with each reading consisting a 30 sec exposure, repeated 10 times (10 scans). Subsequently the protein was concentrated to 50 g/L. FIG. 29 provides calibration data obtained from the buffer (histidine)/protein (adalimumab) system. This is the calibration model for histidine/adalimumab mixture for up to 50 g/L protein. FIG. 30 provides a plot of diafiltration volumes (from 0 to 6 diafiltration volumes) versus histidine concentration (nM) and adalimumab concentrations (g/l) for low concentrations of buffer and protein in a buffer/protein system.

The plots show the change in concentration during diafiltration of the histidine (nM). The plot on the right shows the protein concentration (g/l) during diafiltration and then subsequent ultrafiltration. As expected the concentration of sugar increase during diafiltration reaching a plateau. The protein reaches the target concentration. In this plot (FIG. 29), a model calibrated to 50 g/L was used. The concentration in the plot is lower than expected, due to the model limitation, which was later identified to be related to the ionization of histidine. Models can correlate the ionized state of histidine to the actual total histidine concentration and solution properties.

The data demonstrates the capability to monitor low and high concentration UF/DF operations with a protein and an additional single component. Concentrations can be read every 3 minutes thus providing the ability to monitor concentrations in real time (or near real-time). In the sugar/protein system, very high accuracy was obtained with sugar for all concentrations of protein. In the buffer/protein system, high buffer accuracy was obtained at higher buffer concentrations and lower protein concentrations. The ability to detect and measure volume exclusion effects and Donnan effects is also provided in real-time (or near real-time). Thus Raman spectroscopy is useful as a tool for excipient concentration measurements in protein solutions, and also provides the ability to measure protein concentrations in addition to excipient concentrations to provide process control.

### 6.9 Testing of 2-Component Formulation Buffers with Protein

The methodology of Example 6.5 was applied to formulation buffers containing 2 components, Tris and Acetate, and a protein, Adalimumab. The components were included in the following ranges: Tris 50-160 mM; Acetate 30-130 mM; and Adalimumab 4-15 g/L.

Calibration curves can be obtained as outlined in Example 6.5. The models developed above were used to generate predictions about spectra of mixtures of Tris, Acetate and Adalimumab, in samples prepared according to the concentrations of Table 3.

TABLE 3

| Tris (mM) | Acetate | Ab (g/L) |
|---|---|---|
| 160 | 30 | 4.0 |
| 50 | 130 | 4.0 |
| 50 | 30 | 15.0 |
| 50 | 93 | 8.1 |
| 85 | 30 | 11.5 |
| 99 | 85 | 4.0 |
| 105 | 80 | 9.5 |
| 106 | 59 | 6.2 |
| 100 | 63 | 6.4 |
| 53 | 36 | 14.0 |
| 80 | 72 | 7.4 |
| 102 | 51 | 7.5 |
| 52 | 63 | 11.2 |
| 128 | 52 | 4.8 |
| 128 | 37 | 6.4 |

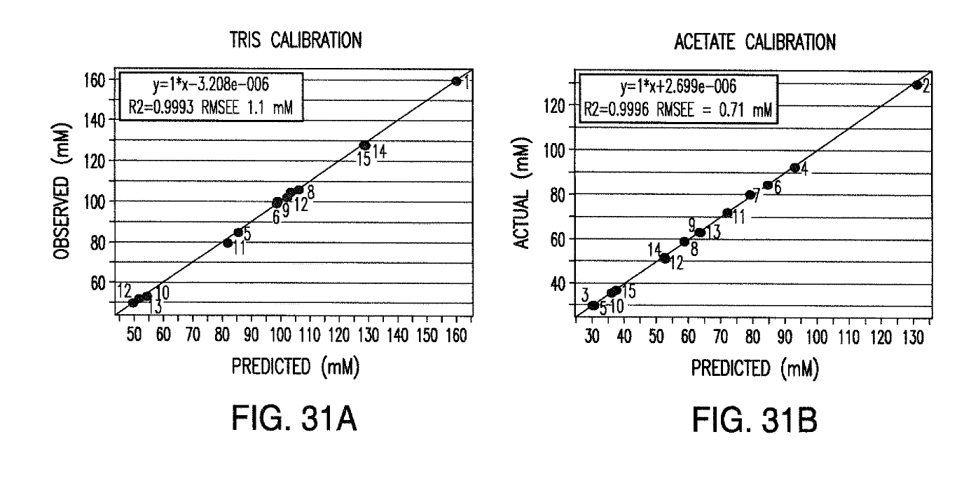

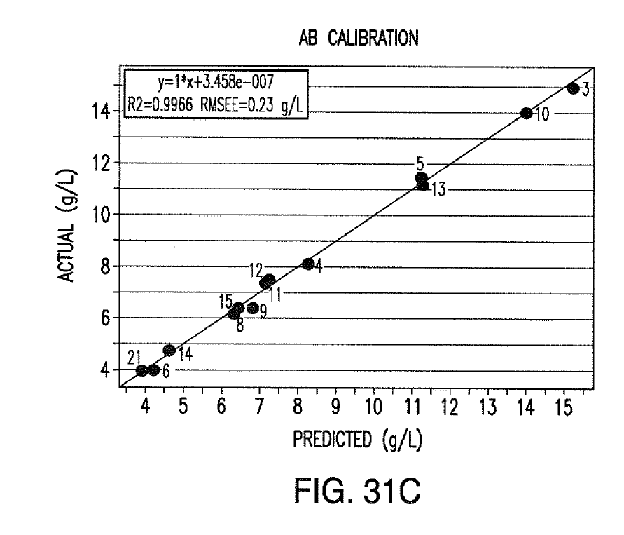

These predictions were compared against the actual spectra to confirm that the model falls within predetermined tolerances. The results are shown in FIG. 31A-C.

### 6.10 Testing of Cell Culture Harvest with Protein

The methodology of Example 6.5 was applied to chemically defined cell culture media harvest containing components, Tween™, and a protein, Adalimumab. The cell culture media was harvested from a cell culture batch, filtered, and loaded onto a protein A column. The protein A column flow through was pooled and then sterile filtered prior to storage and testing.

This methodology would be used to determine the end point of a protein A column load. Filtered cell culture harvest would be applied to a capture column (typically protein A). The current method for monitoring column load output uses A280 absorbance. The culture harvest, however, contains many constituents that absorb light at 280 nm. The A280 absorbance is usually saturated, rendering the A280 method incapable of measuring antibody breakthrough during the column load phase.

The Raman spectrometer offers a specific measurement for antibody in a capture column load output stream (the column flow-through). This test simulates a proposed on-line antibody measurement by spiking various concentrations of purified antibody API drug substance (e.g., Adalimumab) into a pool of protein A flow-through. The API sample used for the spiking experiments contained 0.1% Tween™. During a direct spiking experiment, the Tween™ concentration would change in direct proportion with the antibody, and could be mistaken for antibody during the Raman spectral calibration. To avoid this, the Tween™ was considered an additional component and was spiked independently of the antibody concentrations. The components were therefore included in the following ranges: Tween™ 0.1%-1.0% and Adalimumab 0.1-1.0 g/L.

Calibration curves can be obtained as outlined in Example 6.5. The models developed above were used to generate predictions about spectra of mixtures of Tween™ and Adalimumab, in samples prepared according to the concentrations of Table 4:

TABLE 4

| Adalimumab (g/L) | Tween ™ (%) |
|---|---|
| 1.0 | 0.0 |
| 0.0 | 1.0 |
| 0.6 | 0.6 |
| 1.0 | 0.1 |
| 0.1 | 1.0 |
| 1.0 | 1.0 |
| 0.1 | 0.1 |
| 0.7 | 0.4 |
| 0.1 | 0.3 |
| 0.5 | 0.4 |
| 0.2 | 0.7 |
| 0.8 | 0.3 |

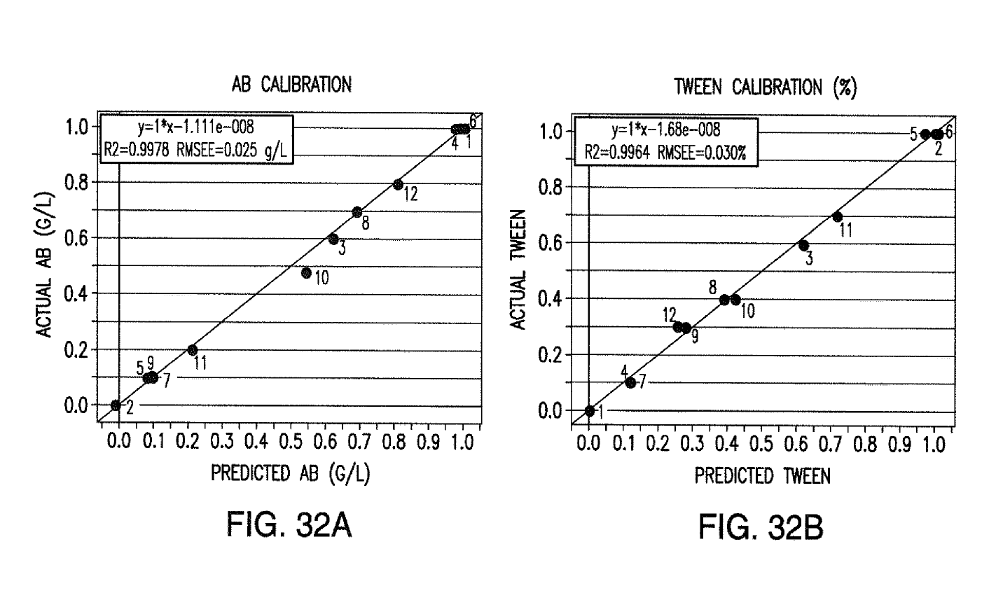

These predictions were compared against the actual spectra to confirm that the model falls within predetermined tolerances. The results are shown in FIG. 32A-B.

### 6.11 Testing of Antibody Aggregate Detection

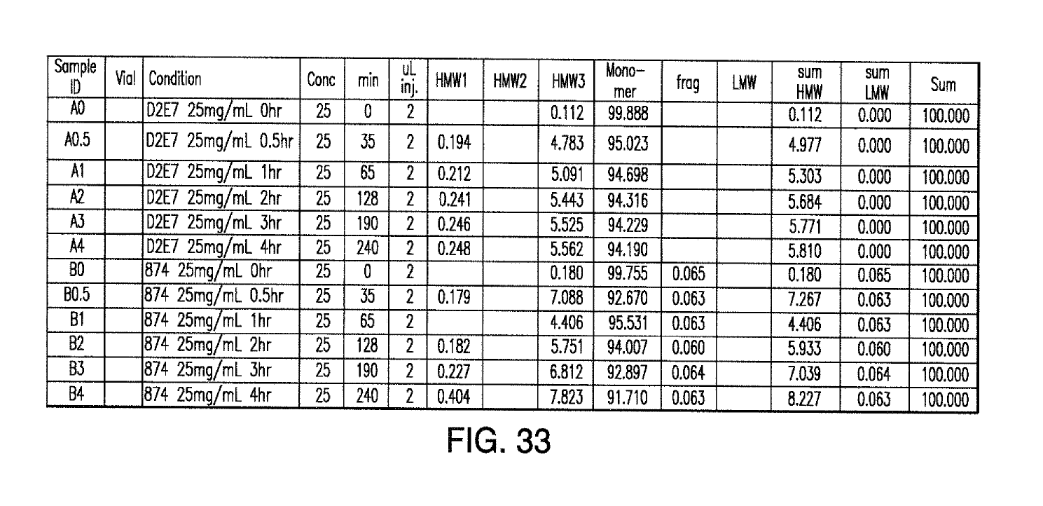

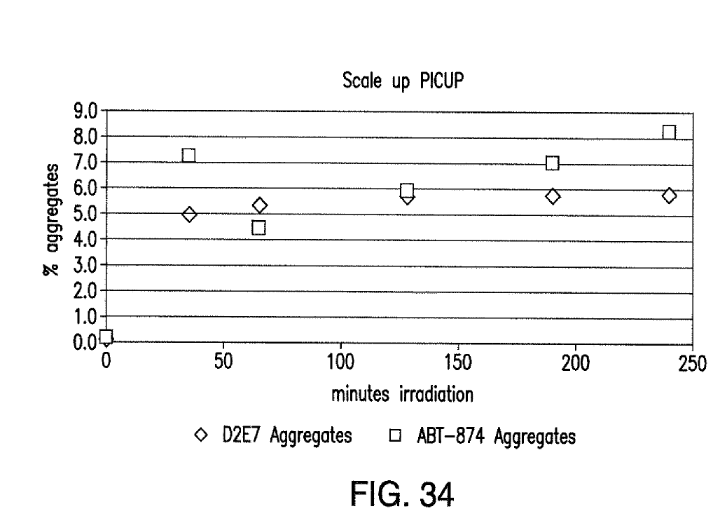

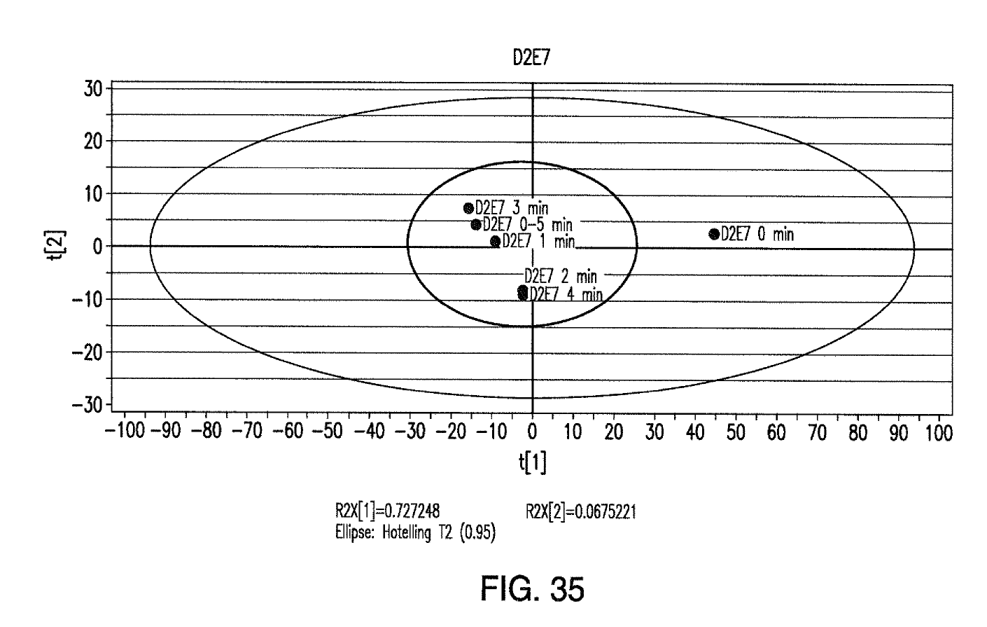

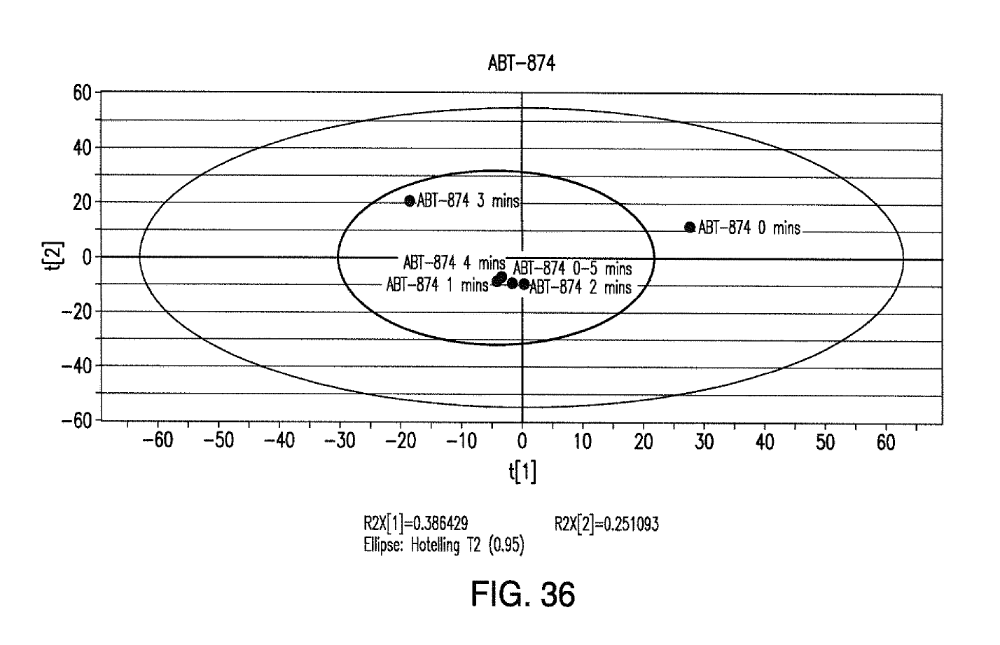

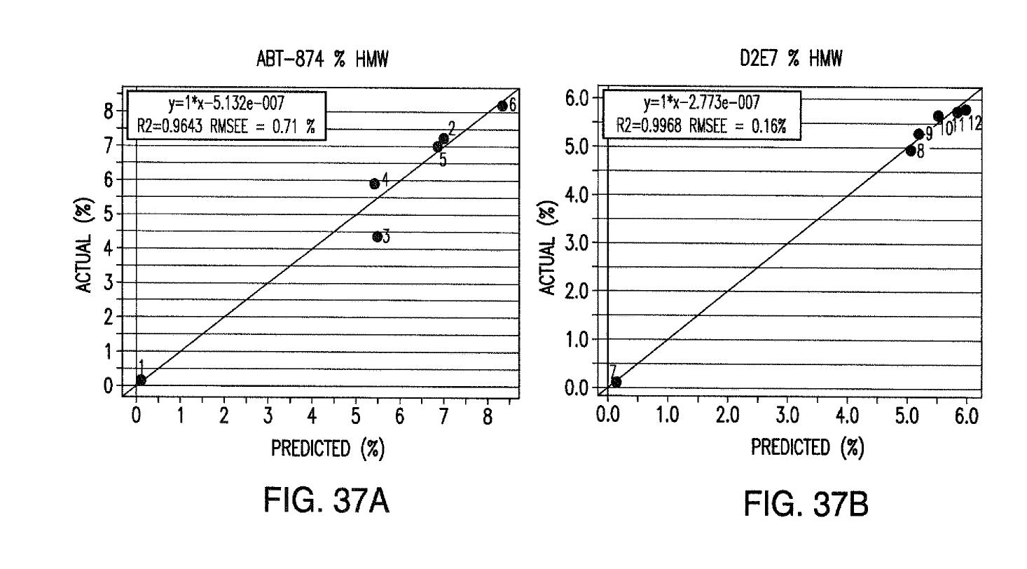

Two antibodies (D2E7 and ABT-874) were separately aggregated using photo induced cross linking of unmodified proteins (PICUP). The antibodies were exposed to the aggregating light source from 0-4 hours (FIGS. 33 and 34) and the aggregation quantified by size exclusion chromatography (SEC). Samples were measured by Raman spectroscopy and the spectra modeled using principal component analysis (PCA) (FIGS. 35 and 36) and partial least squares analysis PLS (FIGS. 37A and 37B). FIGS. 35 and 36 show that aggregated samples have distinct principal component scores and can be discriminated from aggregates using Raman spectroscopy. FIGS. 37A and 37B show some correlation between Raman spectroscopy results and the SEC measurements.

Various publications are cited herein, the contents of which are hereby incorporated in their entireties.

What is claimed is:

1. A method for producing a host cell-protein (HCP) reduced target protein preparation from a sample mixture comprising a target protein and at least one HCP, said method comprising:
(a) performing Raman spectroscopy analysis of said sample mixture;
(b) contacting said sample mixture to a chromatography resin such that the resin is loaded to about 50%-100% of its saturated binding capacity; and
(c) collecting a chromatographic sample; and
(d) performing Raman spectroscopy analysis of said chromatographic sample to identify it as a HCP-reduced target protein preparation.

2. The method of claim 1 wherein the chromatographic resin is selected from the group consisting of affinity chromatographic resin, ion exchange chromatographic resin, and hydrophobic interaction chromatographic resin.

3. The method of claim 1 wherein the target protein is selected from the group consisting of: enzymes; peptide hormones; polyclonal antibodies; human monoclonal antibodies; humanized monoclonal antibodies; chimeric monoclonal antibodies; single chain antibodies; Fab antibody fragments; F(ab′)2 antibody fragments; Fd antibody fragments; Fv antibody fragments; isolated CDRs; diabodies; and immunoadhesions.

4. The method of claim 1 wherein the chromatography resin is packed into a series of fluidly-connected columns separated by fluid conduits comprising inlet and outlet valves, wherein the number of fluidly connected columns is selected from the group consisting of: 2, 3, 4, 5, 6, 7, 8, 9, 10, 11, and 12 columns.

5. The method of claim 1 wherein the sample mixture is contacted to the chromatography resin in order to obtain a residence time selected from the group consisting of up to about 05, up to about 1, up to about 2, up to about 3, up to about 4, up to about 5, up to about 6, up to about 7, up to about 8, up to about 9, up to about 10, up to about 11, and up to about 12 minutes.

6. The method of claim 1 further comprising the steps of equilibrating the chromatographic resin prior to contact with the sample mixture and washing the chromatographic resin after contact with the sample mixture, where the equilibration and wash buffers are identical buffers.

7. The method of claim 1 further comprising chromatography resin wash, elution, and regeneration steps, where such steps can be calculated and programmed in order to maintain the step of contacting the sample to the chromatography resin to be from about 20% to about 80% of the time of the process.
